SEQUENCE LISTING (8 sequences)

SEQ ID NO: 1 - DNA, 13110 bp, Homo sapiens
  CDS (328)..(11301)
ggggaagcagtggccgtgtgagcgtgaggagctgccgccaccgcctgctcctcgtcctcctcgtcctccggggccccagcgtcgtgggccgcgcacggccctggaagagacgtcgcctcgccttcatccgcctctctcaccgcgccgctccctcgtcctgccctgcgggctcaggcggaacccggaacggccgtcctcttcccccgccctccgccgccgcctcctcctcctccttctcggcttcctcctcagccccgggccggagcggggtgtcggcggcggccggttcgggcggcggcgcttggccatgtcgtgtcggggaaggtaatgagccgcagagccccggggtctcggctgagcagcggcggcaccaactattcgcggagctggaatgactggcaacccagaactgatagtgcatcagctgacccaggtaatttaaaatattcttcatccagagatagaggtggttcttcctcttacggactgcaaccttcaaattcagctgtggtgtctcggcaaaggcacgatgataccagagtccacgctgacatacagaatgacgaaaagggtggctacagtgtcaatggaggatctggggaaaatacttatggtcggaagtcgttggggcaagagctgagggttaacaatgtgaccagccctgagttcaccagtgttcagcatggcagtcgtgctttagccaccaaagacatgaggaaatcacaggagagatcgatgtcttattctgatgagtctcgactgtcgaatcttcttcggaggatcacccgggaagacgacagagaccgaagattggctactgtaaagcagttgaaagaatttattcagcaaccagaaaataagctggtactagttaaacaattggataatatcttggctgctgtacatgacgtgcttaatgaaagtagcaaattgcttcaggagttgagacaggagggagcttgctgtcttggccttctttgtgcttctctgagctatgaggctgagaagatcttcaagtggatttttagcaaatttagctcatctgcaaaagatgaagttaaactcctctacttatgtgccacctacaaagcactagagactgtaggagaaaagaaagccttttcatctgtaatgcagcttgtaatgaccagcctgcagtctattcttgaaaatgtggatacaccagaattgctttgtaaatgtgttaagtgcattcttttggtggctcgatgttaccctcatattttcagcactaattttagggatacagttgatatattagttggatggcatatagatcatactcagaaaccttcgctcacgcagcaggtatctgggtggttgcagagtttggagccattttgggtagctgatcttgcattttctactactcttcttggtcagtttctggaagacatggaagcatatgctgaggacctcagccatgtggcctctggggaatcagtggatgaagatgtccctcctccatcagtgtcattaccaaagctggctgcacttctccgggtatttagtactgtggtgaggagcattggggaacgcttcagcccaattcggggtcctccaattactgaggcatatgtaacagatgttctgtacagagtaatgagatgtgtgacggctgcaaaccaggtgtttttttctgaggctgtgttgacagctgctaatgagtgtgttggtgttttgctcggcagcttggatcctagcatgactatacattgtgacatggtcattacatatggattagaccaactggagaattgccagacttgtggtaccgattatatcatctcagtcttgaatttactcacgctgattgttgaacagataaatacgaaactgccatcatcatttgtagaaaaactgtttataccatcatctaaactactattcttgcgttatcataaagaaaaagaggttgttgctgtagcccatgctgtttatcaagcagtgctcagcttgaagaatattcctgttttggagactgcctataagttaatattgggagaaatgacttgtgccctaaacaacctcctacacagtctacaacttcctgaggcctgttctgaaataaaacatgaggcttttaagaatcatgtgttcaatgtagacaatgcaaaatttgtagttaaatttgacctcagtgccctgactacaattggaaatgccaaaaactcactaatagggatgtgggcgctatctccaactgtctttgcacttctgagtaagaatctgatgattgtgcacagtgacctggctgttcacttccctgccattcagtatgctgtgctctacacattgtattctcattgtaccaggcatgatcactttatctctagtagcctcagttctgcctctccttctttgtttgatggagctgtgattagcactgtaactacggctacaaagaaacatttctcaattatattaaatcttctgggaatattacttaagaaagataaccttaaccaggacacgaggaaactgttaatgacttgggctttggaagcagctgttttaatgaggaagtctgaaacatacgcacctttattctctcttccgtctttccataaattttgcaaaggccttttagccaacactctcgttgaagatgtgaatatctgtctgcaggcatgcagcagtctacatgctctgtcctcttccttgccagatgatcttttacagagatgtgtcgatgtttgccgtgttcaactagtgcacagtggaactcgtattcgacaagcatttggaaaactgttgaaatcaattcctttagatgttgtcctaagcaataacaatcacacagaaattcaagaaatttctttagcattaagaagtcacatgagtaaagcaccaagtaatacattccacccccaagatttctctgatgttattagttttattttgtatgggaactctcatagaacagggaaggacaattggttggaaagactgttctatagctgccagagactggataagcgtgaccagtcaacaattccacgcaatctcctgaagacagatgctgtcctttggcagtgggccatatgggaagctgcacaattcactgttctttctaagctgagaaccccactgggcagagctcaagacaccttccagacaattgaaggtatcattcgaagtctcgcagctcacacattaaaccctgatcaggatgttagtcagtggacaactgcagacaatgatgaaggccatggtaacaaccaacttagacttgttcttcttctgcagtatctggaaaatctggagaaattaatgtataatgcatacgagggatgtgctaatgcattaacttcacctcccaaggtcattagaacttttttctataccaatcgccaaacttgtcaggactggctaacgcggattcgactctccatcatgagggtaggattgttggcaggccagcctgcagtgacagtgagacatggctttgacttgcttacagagatgaaaacaaccagcctatctcaggggaatgaattggaagtaaccattatgatggtggtagaagcattatgtgaacttcattgtcctgaagctatacagggaattgctgtctggtcatcatctattgttggaaaaaatcttctgtggattaactcagtggctcaacaggctgaagggaggtttgaaaaggcctctgtggagtaccaggaacacctgtgtgccatgacaggtgttgattgctgcatctccagctttgacaaatcggtgctcaccttagccaatgctgggcgtaacagtgccagcccgaaacattctctgaatggtgaatccagaaaaactgtgctgtccaaaccgactgactcttcccctgaggttataaattatttaggaaataaagcatgtgagttctacatctcaattgccgattgggctgctgtgcaggaatggcagaacgctatccatgacttgaaaaagagtaccagtagcacttccctcaacctgaaagctgacttcaactatataaaatcattaagcagctttgagtctggaaaatttgttgaatgtaccgagcagttagaattgttaccaggagaaaatatcaatctacttgctggaggatcaaaagaaaaaatagacatgaaaaaactgcttcctaacatgttaagtccggatccgagggaacttcagaaatccattgaagttcaattgttaagaagttctgtttgtttggcaactgctttaaacccgatagaacaagatcagaagtggcagtctataactgaaaatgtggtaaagtacttgaagcaaacatcccgcatcgctattggacctctgagactttctactttaacagtttcacagtctttgccagttctaagtaccttgcagctgtattgctcatctgctttggagaacacagtttctaacagactttcaacagaggactgtcttattccactcttcagtgaagctttacgttcatgtaaacagcatgacgtgaggccatggatgcaggcattaaggtatactatgtaccagaatcagttgttggagaaaattaaagaacaaacagtcccaattagaagccatctcatggaattaggtctaacagcagcaaaatttgctagaaaacgagggaatgtgtcccttgcaacaagactgctggcacagtgcagtgaagttcagctgggaaagaccaccactgcacaggatttagtccaacattttaaaaaactatcaacccaaggtcaagtggatgaaaaatgggggcccgaacttgatattgaaaaaaccaaattgctttatacagcaggccagtcaacacatgcaatggaaatgttgagttcttgtgccatatctttctgcaagtctgtgaaagctgaatatgcagttgctaaatcaattctgacactggctaaatggatccaggcagaatggaaagagatttcaggacagctgaaacaggtttacagagctcagcaccaacagaacttcacaggtctttctactttgtctaaaaacatactcactctaatagaactgccatctgttaatacgatggaagaagagtatcctcggatcgagagtgaatctacagtgcatattggagttggagaacctgacttcattttgggacagttgtatcacctgtcttcagtacaggcacctgaagtagccaaatcttgggcagcgttggccagctgggcttataggtggggcagaaaggtggttgacaatgccagtcagggagaaggtgttcgtctgctgcctagagaaaaatctgaagttcagaatctacttccagacactataactgaggaagagaaagagagaatatatggtattcttggacaggctgtgtgtcggccggcggggattcaggatgaagatataacacttcagataactgagagtgaagacaacgaagaagatgacatggttgatgttatctggcgtcagttgatatcaagctgcccatggctttcagaacttgatgaaagtgcaactgaaggagttattaaagtgtggaggaaagttgtagatagaatattcagcctgtacaaactctcttgcagtgcatactttactttccttaaactcaacgctggtcaaattcctttagatgaggatgaccctaggctgcatttaagtcacagagtggaacagagcactgatgacatgattgtgatggccacattgcgcctgctgcggttgctcgtgaagcatgctggtgagcttcggcagtatctggagcacggcttggagacaacacccactgcaccatggaggggaattattccgcaacttttctcacgcttaaaccaccctgaagtgtatgtgcgccaaagtatttgtaaccttctctgccgtgtggctcaagattccccacatctcatattgtatcctgcaatagtgggtaccatatcgcttagtagtgaatcccaggcttcaggaaataaattttccactgcaattccaactttacttggcaatattcaaggagaagaattgctggtttctgaatgtgagggaggaagtcctcctgcatctcaggatagcaataaggatgaacctaaaagtggattaaatgaagaccaagccatgatgcaggattgttacagcaaaattgtagataagctgtcctctgcaaaccccaccatggtattacaggttcagatgctcgtggctgaactgcgcagggtcactgtgctctgggatgagctctggctgggagttttgctgcaacaacacatgtatgtcctgagacgaattcagcagcttgaagatgaggtgaagagagtccagaacaacaacaccttacgcaaagaagagaaaattgcaatcatgagggagaggcacacagctttgatgaagcccatcgtatttgctttggagcatgtgaggagtatcacagcggctcctgcagaaacacctcatgaaaaatggtttcaggataactatggtgatgccattgaaaatgccctagaaaaactgaagactccattgaaccctgcaaagcctgggagcagctggattccatttaaagagataatgctaagtttgcaacagagagcacagaaacgtgcaagttacatcttgcgtcttgaagaaatcagtccatggttggctgccatgactaacactgaaattgctcttcctggggaagtctcagccagagacactgtcacaatccatagtgtgggcggaaccatcacaatcttaccgactaaaaccaagccaaagaaacttctctttcttggatcagatgggaagagctatccttatcttttcaaaggactggaggatttacatctggatgagagaataatgcagttcctatctattgtgaataccatgtttgctacaattaatcgccaagaaacaccccggttccatgctcgacactattctgtaacaccactaggaacaagatcaggactaatccagtgggtagatggagccacacccttatttggtctttacaaacgatggcaacaacgggaagctgccttacaagcacaaaaggcccaagattcctaccaaactcctcagaatcctggaattgtaccccgtcctagtgaactttattacagtaaaattggccctgctttgaaaacagttgggcttagcctggatgtgtcccgtcgggattggcctcttcatgtaatgaaggcagtattggaagagttaatggaggccacacccccgaatctccttgccaaagagctctggtcatcttgcacaacacctgatgaatggtggagagttacgcagtcttatgcaagatctactgcagtcatgtctatggttggatacataattggccttggagacagacatctggataatgttcttatagatatgacgactggagaagttgttcacatagattacaatgtttgctttgaaaaaggtaaaagccttagagttcctgagaaagtaccttttcgaatgacacaaaacattgaaacagcactgggtgtaactggagtagaaggtgtatttaggctttcatgtgagcaggttttacacattatgcggcgtggcagagagaccctgctgacgctgctggaggcctttgtgtacgaccctctggtggactggacagcaggaggcgaggctgggtttgctggtgctgtctatggtggaggtggccagcaggccgagagcaagcagagcaagagagagatggagcgagagatcacccgcagcctgttttcttctagagtagctgagattaaggtgaactggtttaagaatagagatgagatgctggttgtgcttcccaagttggacggtagcttagatgaatacctaagcttgcaagagcaactgacagatgtggaaaaactgcagggcaaactactggaggaaatagagtttctagaaggagctgaaggggtggatcatccttctcatactctgcaacacaggtattctgagcacacccaactacagactcagcaaagagctgttcaggaagcaatccaggtgaagctgaatgaatttgaacaatggataacacattatcaggctgcattcaataatttagaagcaacacagcttgcaagcttgcttcaagagataagcacacaaatggaccttggtcctccaagttacgtgccagcaacagcctttctgcagaatgctggtcaggcccacttgattagccagtgcgagcagctggagggggaggttggtgctctcctgcagcagaggcgctccgtgctccgtggctgtctggagcaactgcatcactatgcaaccgtggccctgcagtatccgaaggccatatttcagaaacatcgaattgaacagtggaagacctggatggaagagctcatctgtaacaccacagtagagcgttgtcaagagctctataggaaatatgaaatgcaatatgctccccagccacccccaacagtgtgtcagttcatcactgccactgaaatgaccctgcagcgatacgcagcagacatcaacagcagacttattagacaagtggaacgcttgaaacaggaagctgtcactgtgccagtttgtgaagatcagttgaaagaaattgaacgttgcattaaagttttccttcatgagaatggagaagaaggatctttgagtctagcaagtgttattatttctgccctttgtacccttacaaggcgtaacctgatgatggaaggtgcagcgtcaagtgctggagaacagctggttgatctgacttctcgggatggagcctggttcttggaggaactctgcagtatgagcggaaacgtcacctgcttggttcagttactgaagcagtgccacctggtgccacaggacttagatatcccgaaccccatggaagcgtctgagacagttcacttagccaatggagtgtatacctcacttcaggaattgaattcgaatttccggcaaatcatatttccagaagcacttcgatgtttaatgaaaggggaatacacgttagaaagtatgctgcatgaactggacggtcttattgagcagaccaccgatggcgttcccctgcagactctagtggaatctcttcaggcctacttaagaaacgcagctatgggactggaagaagaaacacatgctcattacatcgatgttgccagactactacatgctcagtacggtgaattaatccaaccgagaaatggttcagttgatgaaacacccaaaatgtcagctggccagatgcttttggtagcattcgatggcatgtttgctcaagttgaaactgctttcagcttattagttgaaaagttgaacaagatggaaattcccatagcttggcgaaagattgacatcataagggaagccaggagtactcaagttaatttttttgatgatgataatcaccggcaggtgctagaagagattttctttctaaaaagactacagactattaaggagttcttcaggctctgtggtaccttttctaaaacattgtcaggatcaagttcacttgaagatcagaatactgtgaatgggcctgtacagattgtcaatgtgaaaaccctttttagaaactcttgtttcagtgaagaccaaatggccaaacctatcaaggcattcacagctgactttgtgaggcagctcttgatagggctacccaaccaagccctcggactcacactgtgcagttttatcagtgctctgggtgtagacatcattgctcaagtagaggcaaaggactttggtgccgaaagcaaagtttctgttgatgatctctgtaagaaagcggtggaacataacatccagatagggaagttctctcagctggttatgaacagggcaactgtgttagcaagttcttacgacactgcctggaagaagcatgacttggtgcgaaggctagaaaccagtatttcttcttgtaagacaagcctgcagcgggttcagctgcatattgccatgtttcagtggcaacatgaagatctacttatcaatagaccacaagccatgtcagtcacacctcccccacggtctgctatcctaaccagcatgaaaaagaagctgcataccctgagccagattgaaacttctattgcgacagttcaggagaagctagctgcacttgaatcaagtattgaacagcgactcaagtgggcaggtggtgccaaccctgcattggcccctgtactacaagattttgaagcaacgatagctgaaagaagaaatcttgtccttaaagagagccaaagagcaagtcaggtcacatttctctgcagcaatatcattcattttgaaagtttacgaacaagaactgcagaagccttaaacctggatgcggcgttatttgaactaatcaagcgatgtcagcagatgtgttcgtttgcatcacagtttaacagttcagtgtctgagttagagcttcgtttattacagagagtggacactggtcttgaacatcctattggcagctctgaatggcttttgtcagcacacaaacagttgacccaggatatgtctactcagagggcaattcagacagagaaagagcagcagatagaaacggtctgtgaaacaattcagaatctggttgataatataaagactgtgctcactggtcataaccgacagcttggagatgtcaaacatctcttgaaagctatggctaaggatgaagaagctgctctggcagatggtgaagatgttccctatgagaacagtgttaggcagtttttgggtgaatataaatcatggcaagacaacattcaaacagttctatttacattagtccaggctatgggtcaggttcgaagtcaagaacacgttgaaatgctccaggaaatcactcccaccttgaaagaactgaaaacacaaagtcagagtatctataataatttagtgagttttgcatcacccttagtcaccgatgcaacaaatgaatgttcgagtccaacgtcatctgctacttatcagccatccttcgctgcagcagtccggagtaacactggccagaagactcagcctgatgtcatgtcacagaatgctagaaagctgatccagaaaaatcttgctacatcagctgatactccaccaagcaccgttccaggaactggcaagagtgttgcttgtagtcctaaaaaggcagtcagagaccctaaaactgggaaagcggtgcaagagagaaactcctatgcagtgagtgtgtggaagagagtgaaagccaagttagagggccgagatgttgatccgaataggaggatgtcagttgctgaacaggttgactatgtcattaaggaagcaactaatctagataacttggctcagctgtatgaaggttggacagcctgggtgtgaatggcaagacagtagatgagtctggttaagcgaggtcagacatccaccagaatcaactcagcctcaggcatccaaagccacaccacagtcggtggtgatgcaactgggggcttactctgaggaaacctaggaaatctcggtgcactaggaagtgaatcccgcaggacagctgcactcagggatacgcccaacaccatggcctgcaaccccagggtcaagggtgaaggaaagcaaagctcaccgcctgaacacggagattgtctttctgccacagaacagcagcagacgtgtcgggaggttagctgcggaaagaaatcgggatgccgcggagcacagagtgatttggaactccattccacctgaccctgtgtgtacaatccaggaaaaaaacaaaccccactcagaaacagagaaaactggggtcgcgaagaaatcacagccaaggaagatttgatgcattcagattctcgtgtaacacttgttgcttggcaacagtactggttgggttgaccagtaagtagaaaaaggctaaaggctatgcgatatgaatttcagaaatggactgaaaatggagagctatgtaacagatacactacagtagaagaacttacttctgaaatgaagggaaaaaaaccaccccatcgttccctactcctccccaccacttacccgttccccctttacctaatctagtagattagccatctttcaaattcacttttatttcagtccttatatttcatatacttccgtctcgatgctgttaacaacttctgataacatggaaaattcaaggattgtttaaaggtctgatgatcacacacaaaatgtaattccggttatttaagtcatttctgtgattctatcatgtacagtttccagaattgtcactgtgcattcaaaagtaatgaatctaacagacatttgatttaatgtacactcccttttgcttatagtgtgcattttttttggaggtcattcaaattttccctcttctgtgatagctgtagtttctttcatagaaagtagctaatccagtgtaatcttttacctttttaaaaaccaagatagagtatctattagagttttacattgttgatgatagattaacaataaagtgatgttctggtggaggtagactgaaatttttttaattcatgtttttcatttgatacttttaatttacacttagtaaattaaaagttgtttaatttacttggcattttaggacatgtacatgaaacagtgaaaatgagatccaccaacatcttttattaagttcagttattagtctgtgaagtgctttactttttgcacaattttaatagcttgctattcagtaatacattatagtgaattcatgatcaaggtttccttaaatttagcattgcatttcagtactgactgtgtaagctaaattgctgatccaaaataaaaacccagactagaatagggttcttaaaatcaagtatcaatacaaaatagaacacaattaaaatcttaattgttggctgggcacagtggctcacgcctgtaatcccagcactttgggaggccgaggcgggcggatcatgaggttaggagagcgagaccatcctggctaacacggtgaaaccccgtctttactaaaatacaaaaaaaattagccgggtgtggtggcgggcgcctgtagtcccagctactcgggaggctgaggcaggagaatggcgtgaacccaggaggcggagcttgcagtgagccgagattgtgccactgcactccagcctgggcaacagagctagactctgtgtcaaaaataaatgactagat

SEQ ID NO: 2 - protein, 3657 aa, Homo sapiens [sequence omitted]

SEQ ID NO: 3 - DNA, 22 bp, Homo sapiens
agcgttatgtttggtggaagaa

SEQ ID NO: 4 - DNA, 20 bp, Homo sapiens
gcagctgtcaacacagcctc

SEQ ID NO: 5 - DNA, 19 bp, Homo sapiens
gatgtgtcgatgtttgccg

SEQ ID NO: 6 - DNA, 21 bp, Homo sapiens
ttagcacatccctcgtatgca

SEQ ID NO: 7 - protein, 15 aa, Homo sapiens
CDNLAQLYEGWTAWV

SEQ ID NO: 8 - protein, 10 aa, Artificial Sequence
  Description of Artificial Sequence A His tag sequence containing six histidine residues
MRGSHHHHHH

(19) United States
(12) Patent Application Publication
(10) Pub. No.: US 2004/0137592 A1
(43) Pub. Date: Jul. 15, 2004

(54) Novel SMG-1

(75) Inventor: Shigeo Ohno, Tokyo (JP)

Correspondence Address: HESLIN ROTHENBERG FARLEY & MESITI PC, 5 COLUMBIA CIRCLE, ALBANY, NY, 12203, US

(73) Assignee: Japan Science and Technology Agency, Saitama (JP)

(21) Appl. No.: 10/720,460

(22) Filed: Nov. 24, 2003

Related U.S. Application Data
(63) Continuation-in-part of application No. PCT/JP01/10234, filed on Nov. 22, 2001.

(30) Foreign Application Priority Data
May 24, 2001 (JP) 2001-156088

(51) Int. Cl. C07H 21/04; C12N 9/10

(52) U.S. Cl. 435/193; 435/69.1; 435/320.1; 435/325; 536/23.2

(57) ABSTRACT

A novel polypeptide and a novel polynucleotide encoding the same are disclosed.

The polypeptide is SMG-1, a protein included in the phosphatidyl inositol kinase related kinase family, and is useful in constructing a screening system for agents of treating and/or preventing a disease caused by a premature translation termination codon generated by a nonsense mutation.

### CROSS REFERENCE TO RELATED APPLICATION

This is a continuation-in-part application of International Application No. PCT/JP01/10234 filed on Nov. 22, 2001.

### TECHNICAL FIELD

The present invention relates to SMG-1.

### BACKGROUND ART

In eukaryotes, although a promoter site is the same as that of a normal gene, a nonsense mutation mRNA, in which a codon in the inherent translational region of a gene is changed to a stop codon, is recognized and specifically degraded. One such mechanism for specific degradation is nonsense mediated mRNA decay (NMD). As the genes relating to this mechanism, three genes (UPF1, UPF2, and UPF3) have been reported from yeast and seven genes (SMG-1 to SMG-7) from Caenorhabditis elegans. In mutant organisms of these genes, it has also been reported that the specific degradation of nonsense mutation mRNA is suppressed. In this connection, yeast UPF1 protein and C. elegans SMG-2 protein have a high homology between their amino acid sequences. Further, as a human gene and mouse gene having a high homology of the base sequence with the yeast UPF1 gene, Rent1/HUPF1 (hereinafter referred to simply as “human UPF1”) has been isolated. It is shown that this gene complements the functions of UPF-1 in UPF-1 mutant yeast. Further, when expressing a mutant human UPF1 protein wherein the 844th arginine is mutated to cysteine in animal cells, a suppression of the specific degradation of nonsense mutated mRNA is seen. In this connection the mutants of these genes are not lethal, and are not believed to be genes required for survival.

The UPF1/SMG-2 protein has a Zn finger motif and RNA helicase-like structure and is believed to function as a unit of the complex for degradation of mRNA. Further, other genes are believed to regulate, for example, the activity or location of this enzyme. In C. elegans, it has been reported that the SMG-2 protein is phosphorylated, and that in C. elegans of mutants of the genes of SMG-1, SMG-3, or SMG-4, the SMG-2 protein is not phosphorylated. Further, the base sequence of the cDNA of C. elegans SMG-1 has been reported. The SMG-1 protein has a kinase domain having a high homology with the kinase domain conserved as the family of the group of serine/threonine kinases known as phosphatidyl inositol kinase related kinases (PIKK) and is considered to be PIKK family. Further, a sequence believedw to be fruit-fly SMG-1 has been reported from the base sequence of the fruit-fly genome gene. However, the base sequence of the SMG-1 gene of mammals, including humans, and the amino acid sequence of the SMG-1 protein encoding the same have not been elucidated.

### DISCLOSURE OF INVENTION

The present inventor engaged in intensive search with the object of obtaining a novel phosphatidyl inositol kinase (PIK) related kinase (PIKK) and, as a result, obtained a novel human SMG-1 protein and DNA encoding the same. Further, the present inventor showed that the human SMG-1 has an autophosphorylation activity and an activity of phosphorylating UPF1/SMG-2, and further immunoprecipitates together with UPF1/SMG-2, UPF2, and UPF3. From these facts, the present inventor proved that the human SMG-1 is a member of the surveillance complex which triggers the NMD, and that SMG-1 is actually essential for NMD in mammalian cells using point mutations of SMG-1. Further, the present inventor newly discovered that NMD can be suppressed by inhibiting human SMG-1. The present invention is based on these findings.

Therefore, the object of the present invention is to provide a novel phosphatidyl inositol kinase (PIK) related kinase (PIKK) and a novel polynucleotide encoding the same.

The present invention relates to (1) a polypeptide comprising an amino acid sequence consisting of 129th to 3657th amino acids in the amino acid sequence of SEQ ID NO: 2, or (2) a polypeptide exhibiting an SMG-1 activity and comprising an amino acid sequence in which one or plural amino acids are deleted, substituted, and/or inserted at one or plural positions in an amino acid sequence consisting of 129th to 3657th amino acids in the amino acid sequence of SEQ ID NO: 2.

Further, the present invention relates to a polypeptide exhibiting an SMG-1 activity and comprising an amino acid sequence having a 90% or more homology, with an amino acid sequence consisting of 129th to 3657th amino acids in the amino acid sequence of SEQ ID NO: 2, with an amino acid sequence consisting of 1st to 3657th amino acids in the amino acid sequence of SEQ ID NO: 2, or with an amino acid sequence consisting of 107th to 3657th amino acids in the amino acid sequence of SEQ ID NO: 2.

Further, the present invention relates to a polypeptide consisting of the amino acid sequence of SEQ ID NO: 2.

Further, the present invention relates to a polynucleotide encoding any one of these polypeptides.

Further, the present invention relates to an expression vector comprising the polynucleotide.

Further, the present invention relates to a cell transfected with the expression vector.

Further, the present invention relates to an antibody or a fragment thereof, which binds to the above polypeptide.

Further, the present invention relates to a knock-out non-human animal wherein an expression of a gene encoding the above polypeptide is partially or completely suppressed.

Further, the present invention relates to a method for screening a substance which modifies an SMG-1 activity of the above polypeptide, comprising the steps of: bringing into contact (1) the polypeptide, (2) Upf1/SMG-2, a fragment thereof capable of being phosphorylated, or a fusion polypeptide comprising Upf1/SMG-2 or the fragment thereof, and (3) a substance to be tested; and carrying out phosphorylation under the conditions that the polypeptide is brought into contact with Upf1/SMG-2, the fragment thereof, or the fusion polypeptide, and analyzing whether or not Upf1/SMG-2, the fragment thereof, or the fusion polypeptide is phosphorylated.

Further, the present invention relates to a method for screening a substance which modifies an SMG-1 activity of the above polypeptide, comprising the steps of: bringing (1) the polypeptide into contact with (2) a substance to be tested; and carrying out phosphorylation under the conditions that the polypeptide is brought into contact with the substance to be tested, and analyzing whether or not the polypeptide is autophosphorylated.

Further, the present invention relates to an agent for suppressing nonsense-mediated mRNA decay, comprising, as an active ingredient, a substance which is obtained by one of these screening methods and modifies an SMG-1 activity of the above polypeptide.

Further, the present invention relates to an agent for suppressing nonsense-mediated mRNA decay, comprising as an active ingredient, an inhibitor of a phosphatidyl inositol kinase related kinase.

Further, the present invention relates to an agent for treating and/or preventing a disease caused by a premature translation termination codon generated by a nonsense mutation, comprising, as an active ingredient, a substance which is obtained by the above screening method and modifies an SMG-1 activity of the above polypeptide.

Further, the present invention relates to an agent for treating and/or preventing a disease caused by a premature translation termination codon generated by a nonsense mutation, comprising as an active ingredient, an inhibitor of a phosphatidyl inositol kinase related kinase.

Further, the present invention relates to an agent for suppressing nonsense, comprising as an active ingredient, (1) an SMG-1-acitivity-deficient mutant, or an inhibitor of a phosphatidyl inositol kinase related kinase, and (2) an aminoglycoside antibiotic.

Further, the present invention relates to an agent for suppressing nonsense, comprising, as an active ingredient, an SMG-1-acitivity-deficient mutant, or an inhibitor of a phosphatidyl inositol kinase related kinase.

Further, the present invention relates to an agent for promoting nonsense-mediated mRNA decay, comprising as an active ingredient, (1) the above polypeptide, (2) a substance which promotes an SMG-1 activity of the polypeptide, or (3) the above polynucleotide.

Further, the present invention relates to a method for identifying a nonsense mutation point in a gene, comprising the steps of: culturing a cell to be tested which is obtained from a subject to be tested and may contain a gene having a nonsense mutation by a premature translation termination codon, in the presence of an inhibitor of an SMG-1 activity; and analyzing molecular weight of a polypeptide derived from the gene in the cultured cell.

Further, the present invention relates to a method for detecting a gene having a nonsense mutation, comprising the steps of: culturing at least two groups of cells to be tested which are obtained from a subject to be tested and may contain a gene having a nonsense mutation by a premature-translation termination codon, in the presence of an inhibitor of an SMG-1 activity and in the absence thereof, respectively; and detecting a presence or absence of the difference of an amount of mRNA derived from the gene in the cultured cells.

The term “SMG-1 activity” as used herein means an activity of phosphorylating Upf1/SMG-2 [Sun, X. et al., Proc. Natl. Acad. Sci. USA, 95, 10009-10014 (1998); and Bhattacharya, A. et al., RNA, 6, 1226-1235 (2000)].

### BRIEF DESCRIPTION OF DRAWINGS

FIG. 1 is a drawing showing the relationship between cDNA clones obtained in Example 1 and the novel base sequences and open reading frames obtained therefrom.

FIG. 2 is a drawing showing the results of a comparison between the human SMG-1 of the present invention and known proteins.

FIG. 3 is a photograph, instead of a drawing, showing the results of autoradiography detection of the mRNA of human SMG-1 in various human cell lines.

FIG. 4 is a drawing showing antigen sites used for preparing antibodies against human SMG-1.

FIG. 5 is a photograph, instead of a drawing, showing the results of Western blotting for the HeLa cell lysate.

FIG. 6 is a photograph, instead of a drawing, showing the results of Western blotting for various animal cell lysates.

FIG. 7 is a photograph, instead of a drawing, showing the results of Western blotting for cell lysates derived from various animal tissues.

FIG. 8 is a photograph, instead of a drawing, showing results of Western blotting and the results of confirmation of protein kinase activity, with respect to the immunoprecipitate derived from the HeLa cell lysate.

FIG. 9 is a photograph, instead of a drawing, showing the expression of 6H-hSMG-1 and 6H-hSMG-1 (DA) and results of confirmation of in vitro protein kinase activity.

FIG. 10 is a drawing schematically showing the structure of a reporter gene plasmid.

FIG. 11 is a photograph, instead of a drawing, showing the results of evaluation of the amount of accumulation of reporter mRNA by Northern blotting.

FIG. 12 is a photograph, instead of a drawing, showing representative examples of the results of confirmation of the effects of 6H-hSMG-1 and 6H-hSMG-1 (DA) on the accumulation of reporter mRNA.

FIG. 13 is a graph of the results of statistical processing of the results of confirmation of the effects of 6H-hSMG-1 and 6H-hSMG-1 (DA) on the accumulation of reporter mRNA.

FIG. 14 is a photograph, instead of a drawing, showing representative examples of the results of confirmation of the effects of 6H-hSMG-1 and 6H-hSMG-1 (DA) on the accumulation of reporter mRNA in the presence of doxycycline where BGG-WT was used as a reporter mRNA.

FIG. 15 is a graph of the results of statistical processing of the results of a graphing of the results shown in FIG. 14.

FIG. 16 is a photograph, instead of a drawing, showing representative examples of the results of confirmation of the effects of 6H-hSMG-1 and 6H-hSMG-1 (DA) on the accumulation of mRNA in the presence of doxycycline where BGG-39PTC was used as the reporter mRNA.

FIG. 17 is a graph of the results of statistical processing of the results of a graphing of the results shown in FIG. 14.

FIG. 18 is a photograph, instead of a drawing, showing the results of confirmation of the phosphorylation of full-length hUpf1/SMG-2 fusion protein by 6H-hSMG-1.

FIG. 19 is a drawing schematically showing the structure of hUpf1/SMG-2 partial fragments used in Example 9(2).

FIG. 20 is a photograph, instead of a drawing, showing the results of confirmation of the phosphorylation in fusion proteins of hUpf1/SMG-2 partial fragments by 6H-hSMG-1.

FIG. 21 is a drawing schematically showing the structure of hUpf1/SMG-2 partial peptides used in Example 9(3).

FIG. 22 is a photograph, instead of a drawing, showing the results of confirmation of the phosphorylation in fusion proteins of hUpf1/SMG-2 partial peptides by 6H-hSMG-1.

FIG. 23 is a photograph, instead of a drawing, showing the results of confirmation of the phosphorylation of hUpf1/SMG-2 in the presence of okadaic acid in vivo.

FIG. 24 is a photograph, instead of a drawing, showing the results of confirmation of the phosphorylation of hUpf1/SMG-2 in vivo using alkaline phosphatase.

FIG. 25 is a photograph, instead of a drawing, showing the results of confirmation of the phosphorylation of HA-hUpf1/SMG-2 in the case of an overexpression of 6H-hSMG-1 or 6H-hSMG-1 (DA).

FIG. 26 is a graph showing the inhibitory effect of wortmannin on the kinase activity of 6H-hSMG-1.

FIG. 27 is a graph showing the inhibitory effect of caffeine on the kinase activity of 6H-hSMG-1.

FIG. 28 is a photograph, instead of a drawing, showing the results of confirmation of the inhibition by SMG-1 inhibitors on the phosphorylation of hUpf1/SMG-2 in the cell.

FIG. 29 is a photograph, instead of a drawing, showing the stabilization of the endogenous PTC containing BGG gene product by SMG-1 inhibitors.

FIG. 30 is a drawing schematically showing the structure of the p53 gene and the PTC mutations in the cell lines calu6 and N417.

FIG. 31 is a photograph, instead of a drawing, showing the stabilization of the endogenous PTCp53 gene product by the SMG-1 inhibitor (wortmannin).

FIG. 32 is a photograph, instead of a drawing, showing the stabilization of the endogenous PTCp53 gene product by various concentrations of SMG-1 inhibitors (wortmannin or caffeine).

### BEST MODE FOR CARRYING OUT THE INVENTION

The present invention will be explained in detail hereinafter.

The present inventor found a novel PIKK consisting of 3657 amino acid residues, i.e., human SMG-1. The amino acid sequence thereof is the sequence consisting of the 1st to 3657th amino acids in the amino acid sequence of SEQ ID NO: 2. Further, the present inventor found that a C-terminal fragment consisting of the 107th to 3657th amino acid residues in the novel protein and another C-terminal fragment consisting of the 129th to 3657th amino acid residues therein also exhibit an SMG-1 activity as well as the novel polypeptide. The present invention is based on these findings.

The polypeptide of the present invention includes

(1) a polypeptide comprising the amino acid sequence consisting of the 129th to 3657th amino acids in the amino acid sequence of SEQ ID NO: 2;

(2) a polypeptide exhibiting an SMG-1 activity and comprising an amino acid sequence in which one or plural amino acids are deleted, substituted, and/or inserted at one or plural positions in the amino acid sequence consisting of the 129th to 3657th amino acids in the amino acid sequence of SEQ ID NO: 2 (hereinafter referred to as a functionally equivalent mutant); and

(3) a polypeptide exhibiting an SMG-1 activity and comprising an amino acid sequence having a 90% or more homology, with the amino acid sequence consisting of the 129th to 3657th amino acids in the amino acid sequence of SEQ ID NO: 2, with the amino acid sequence consisting of the 1st to 3657th amino acids in the amino acid sequence of SEQ ID NO: 2, or with the amino acid sequence consisting of the 107th to 3657th amino acids in the amino acid sequence of SEQ ID NO: 2 (hereinafter referred to as a homologous polypeptide).

The “polypeptide comprising the amino acid sequence consisting of the 129th to 3657th amino acids in the amino acid sequence of SEQ ID NO: 2” as the polypeptide of the present invention is not limited, so long as it is a polypeptide comprising the amino acid sequence consisting of the 129th to 3657th amino acids in the amino acid sequence of SEQ ID NO: 2, and exhibiting an SMG-1 activity. It includes, for example,

(1a) a polypeptide having the base sequence consisting of the 107th to 3657th amino acids in the amino acid sequence of SEQ ID NO: 2;

(1b) a fusion polypeptide having an amino acid sequence in which an appropriate marker sequence or the like is added to the N-terminus and/or the C-terminus of the amino acid sequence consisting of the 107th to 3657th amino acids in the amino acid sequence of SEQ ID NO: 2, and exhibiting an SMG-1 activity;

(1c) a polypeptide consisting of the amino acid sequence of SEQ ID NO: 2;

(1d) a fusion polypeptide having an amino acid sequence in which an appropriate marker sequence or the like is added to the N-terminus and/or the C-terminus of the amino acid sequence of SEQ ID NO: 2, and exhibiting an SMG-1 activity;

(1e) a polypeptide having the base sequence consisting of the 129th to 3657th amino acids in the amino acid sequence of SEQ ID NO: 2; and

(1f) a fusion polypeptide having an amino acid sequence in which an appropriate marker sequence or the like is added to the N-terminus and/or the C-terminus of the amino acid sequence consisting of the 129th to 3657th amino acids in the amino acid sequence of SEQ ID NO: 2, and exhibiting an SMG-1 activity.

A method for confirming whether or not a polypeptide to be tested “exhibits an SMG-1 activity” as used herein is not particularly limited. It may be confirmed, for example, by carrying out phosphorylation under the conditions that the test polypeptide is brought into contact with Upf1/SMG-2 (for example, human Upf1/SMG-2), a fragment thereof capable of being phosphorylated, or a fusion polypeptide comprising Upf1/SMG-2 or the fragment thereof, and then analyzing whether or not Upf1/SMG-2, the fragment thereof, or the fusion polypeptide is phosphorylated, more particularly, for example, by the method described in Example 9(1).

The above polypeptide (1a), i.e., “the polypeptide having the base sequence consisting of the 107th to 3657th amino acids in the amino acid sequence of SEQ ID NO: 2” is a novel protein consisting of 3551 amino acid residues and exhibiting an SMG-1 activity. The polypeptide (1a) corresponds to a partial polypeptide of the above polypeptide (1c), i.e., “the polypeptide consisting of the amino acid sequence of SEQ ID NO: 2”.

The polypeptide (1c) is a novel protein having a molecular weight of approximately 430 kDa, and referred to as “p430” in EXAMPLES.

The above polypeptide (1e), i.e., “the polypeptide having the base sequence consisting of the 129th to 3657th amino acids in the amino acid sequence of SEQ ID NO: 2” is a novel protein consisting of 3529 amino acid residues and exhibiting an SMG-1 activity. The polypeptide (1e) corresponds to a partial polypeptide of the polypeptide (1c), and is a novel protein having a molecular weight of approximately 400 kDa, and referred to as “p400” in EXAMPLES.

As the marker sequence in the polypeptide of the present invention, for example, a sequence for easily carrying out confirmation of polypeptide expression, confirmation of intracellular localization thereof, purification thereof, or the like may be used. As the sequence, there may be mentioned, for example, the FLAG tag, the hexa-histidine tag, the hemagglutinin tag, the myc epitope, or the like.

The functionally equivalent mutant of the present invention is not particularly limited, so long as it is a polypeptide comprising an amino acid sequence in which one or plural (preferably 1 to 10, more preferably 1 to 7, most preferably 1 to 5) amino acids, such as one to several amino acids, are deleted, substituted, and/or inserted at one or plural positions in the amino acid sequence consisting of the 129th to 3657th amino acids in the amino acid sequence of SEQ ID NO: 2, and exhibiting an SMG-1 activity. Further, an origin of the functionally equivalent mutant is not limited to a human.

The functionally equivalent mutant of the present invention includes, for example, human mutants of the polypeptide having the amino acid sequence consisting of the 129th to 3657th amino acids in the amino acid sequence of SEQ ID NO: 2, and functionally equivalent mutants derived from organisms other than human (such as simian, mouse, rat, hamster, or dog). As the functionally equivalent mutants derived from organisms other than human, there may be mentioned, a simian native polypeptide having a molecular weight of 400 kDa or 430 kDa, a rat native polypeptide having a molecular weight of 400 kDa or 430 kDa, or a mouse native polypeptide having a molecular weight of 400 kDa or 430 kDa, as shown in Example 5.

Further, the functionally equivalent mutant of the present invention includes polypeptides prepared using polynucleotides obtained by artificially modifying polynucleotides encoding these native polypeptides (i.e., human mutants or functionally equivalent mutants derived from organisms other than human) or polynucleotides encoding the polypeptide consisting of the 129th to 3657th amino acids in the amino acid sequence of SEQ ID NO: 2 by genetic engineering techniques.

Human mutants of the polypeptide consisting of the 129th to 3657th amino acids in the amino acid sequence of SEQ ID NO: 2 or functionally equivalent mutants derived from organisms other than a human may be obtained by those skilled in the art in accordance with the information of a base sequence (for example, the base sequence consisting of 712th to 11301st bases in the base sequence of SEQ ID NO: 1) of a polynucleotide encoding the polypeptide having the amino acid sequence consisting of the 129th to 3657th amino acids in the amino acid sequence of SEQ ID NO: 2. In this connection, genetic engineering techniques may be generally performed in accordance with known methods (for example, Sambrook, J. et al., “Molecular Cloning-A Laboratory Manual”, Cold Spring Harbor Laboratory, NY, 1989).

For example, an appropriate probe or appropriate primers are designed in accordance with the information of a base sequence of a polynucleotide encoding the polypeptide having the amino acid sequence consisting of the 129th to 3657th amino acids in the amino acid sequence of SEQ ID NO: 2. A polymerase chain reaction (PCR) method (Saiki, R. K. et al., Science, 239, 487-491, 1988) or a hybridization method is carried out using a sample (for example, total RNA or an mRNA fraction, a cDNA library, or a phage library) prepared from an organism (for example, a mammal such as human, simian, mouse, rat, hamster, or dog) of interest and the primers or the probe to obtain a polynucleotide encoding the polypeptide. A desired polypeptide may be obtained by expressing the resulting polynucleotide in an appropriate expression system and confirming that the expressed polypeptide exhibits an SMG-1 activity by, for example, the method described in Example 9(1).

Further, the polypeptide artificially modified by genetic engineering techniques may be obtained by, for example, the following procedure. A gene encoding the polypeptide may be obtained by a conventional method, for example, site-directed mutagenesis (Mark, D. F. et al., Proc. Natl. Acad. Sci. USA, 81, 5662-5666, 1984). A desired polypeptide may be obtained by expressing the resulting polynucleotide in an appropriate expression system and confirming that the expressed polypeptide exhibits an SMG-1 activity by, for example, the method described in Example 9(1).

The homologous polypeptide of the present invention is not particularly limited, so long as it is a polypeptide comprising an amino acid sequence having a 90% or more homology, with the amino acid sequence consisting of the 129th to 3657th amino acids in the amino acid sequence of SEQ ID NO: 2, with the amino acid sequence consisting of the 1st to 3657th amino acids in the amino acid sequence of SEQ ID NO: 2, or with the amino acid sequence consisting of the 107th to 3657th amino acids in the amino acid sequence of SEQ ID NO: 2, and exhibiting an SMG-1 activity. The homologous polypeptide of the present invention may comprise an amino acid sequence having preferably a 95% or more homology, more preferably a 98% or more homology, most preferably a 99% or more homology, with respect to the amino acid sequence consisting of the 129th to 3657th amino acids in the amino acid sequence of SEQ ID NO: 2, the amino acid sequence consisting of the 1st to 3657th amino acids in the amino acid sequence of SEQ ID NO: 2, or the amino acid sequence consisting of the 107th to 3657th amino acids in the amino acid sequence of SEQ ID NO: 2. As the homologous polypeptide of the present invention, a polypeptide having an amino acid sequence having a 90% or more homology (preferably a 95% or more homology, more preferably a 98% or more homology, most preferably a 99% or more homology), with the amino acid sequence consisting of the 129th to 3657th amino acids in the amino acid sequence of SEQ ID NO: 2, with the amino acid sequence consisting of the 1st to 3657th amino acids in the amino acid sequence of SEQ ID NO: 2, or with the amino acid sequence consisting of the 107th to 3657th amino acids in the amino acid sequence of SEQ ID NO: 2, and exhibiting an SMG-1 activity is preferable.

The term “homology” as used herein means a value obtained by BLAST [Basic local alignment search tool; Altschul, S. F. et al., J. Mol. Biol., 215, 403-410, (1990)].

Further, the polypeptide of the present invention includes a polypeptide obtained by bringing mammalian cells or disrupted cells (such as cell lysate) into contact with an antibody specific for SMG-1 to form an immunocomplex (such as immunoprecipitate) and then removing the antibody from the immunocomplex. As the polypeptide, there may be mentioned, for example, a human, simian, rat, or mouse native polypeptide having a molecular weight of 400 kDa or 430 kDa.

The polynucleotide of the present invention is not particularly limited, so long as it encodes the polypeptide of the present invention. As the polynucleotide of the present invention, there may be mentioned, for example, a polynucleotide comprising the base sequence consisting of the 712th to 11301st bases in the base sequence of SEQ ID NO: 1, and

(i) the polynucleotide having the base sequence consisting of the 646th to 11301st bases in the base sequence of SEQ ID NO: 1 [and encoding the above polypeptide (1a) of the present invention];

(ii) the polynucleotide having the base sequence consisting of the 328th to 11301st bases in the base sequence of SEQ ID NO: 1 [and encoding the above polypeptide (1c) of the present invention]; or

(iii) the polynucleotide having the base sequence consisting of the 712th to 11301st bases in the base sequence of SEQ ID NO: 1 [and encoding the above polypeptide (1e) of the present invention]

is preferable. In this connection, the term “polynucleotide” as used herein includes both DNA and RNA.

A method for producing the polynucleotide of the present invention is not particularly limited, but there may be mentioned, for example, (1) a method using PCR, (2) a method using conventional genetic engineering techniques (i.e., a method for selecting a transformant comprising a desired cDNA from strains transformed with a cDNA library), or (3) a chemical synthesis method. These methods will be explained in this order hereinafter.

In the method using PCR of the item (1), the polynucleotide of the present invention may be produced, for example, by the following procedure.

mRNA is extracted from human cells or tissue capable of producing-the polypeptide of the present invention. A pair of primers, between which full-length mRNA corresponding to the polypeptide of the present invention or a partial region of the mRNA is located, is synthesized on the basis of the base sequence of a polynucleotide encoding the polynucleotide of the present invention. Full-length cDNA encoding the polypeptide of the present invention or a part of the cDNA may be obtained by performing a reverse transcriptase-polymerase chain reaction (RT-PCR) using the extracted mRNA as a template.

More particularly, total RNA containing mRNA encoding the polypeptide of the present invention is extracted by a known method from cells or tissue capable of producing the polypeptide of the present invention. As an extraction method, there may be mentioned, for example, a guanidine thiocyanate-hot phenol method, a guanidine thiocyanate-guanidine hydrochloride method, or a guanidine thiocyanate-cesium chloride method. The guanidine thiocyanate-cesium chloride method is preferably used. The cells or tissue capable of producing the polypeptide of the present invention may be identified, for example, by a northern blotting method using a polynucleotide or a part thereof encoding the polypeptide of the present invention or a western blotting method using an antibody specific for the polypeptide of the present invention.

Next, the extracted mRNA is purified. Purification of the mRNA may be made in accordance with a conventional method. For example, the mRNA may be purified by adsorption and elution using an oligo(dT)-cellulose column. The mRNA may be further fractionated by, for example, a sucrose density gradient centrifugation, if necessary. Alternatively, commercially available extracted and purified mRNA may be used without carrying out the extraction of the mRNA.

Next, the first-strand cDNA is synthesized by carrying out a reverse transcriptase reaction of the purified mRNA in the presence of a random primer, an oligo dT primer, and/or a custom primer. This synthesis may be carried out in accordance with a conventional method. The resulting first-strand cDNA is subjected to PCR using two primers between which a full-length or a partial region of the polynucleotide of interest is located, thereby amplifying the cDNA of interest. The resulting DNA is fractionated by, for example, an agarose gel electrophoresis. The DNA fragment of interest may be obtained by carrying out a digestion of the DNA with restriction enzymes and subsequent ligation, if necessary.

In the method using conventional genetic engineering techniques of the item (2), the polynucleotide of the present invention may be produced, for example, by the following procedure.

First, single-stranded cDNA is synthesized by using reverse transcriptase from mRNA prepared by the above-mentioned PCR method as a template, and then double-stranded cDNA is synthesized from the single-stranded cDNA. As this method, there may be mentioned, for example, an S1 nuclease method (Efstratiadis, A. et al., Cell, 7, 279-288, 1976), a Land method (Land, H. et al., Nucleic Acids Res., 9, 2251-2266, 1981), an O. Joon Yoo method (Yoo, O. J. et al., Proc. Natl. Acad. Sci. USA, 79, 1049-1053, 1983), and an Okayama-Berg method (Okayama, H. and Berg, P., Mol. Cell. Biol., 2, 161-170, 1982).

Next, a recombinant plasmid comprising the double-stranded cDNA is prepared and introduced into an Escherichia coli strain, such as DH 5α, HB101, or JM109, thereby transforming the strain. A transformant is selected using a drug resistance against, for example, tetracycline, ampicillin, or kanamycin as a marker. When the host cell is E. coli, transformation of the host cell may be carried out, for example, by the method of Hanahan (Hanahan, D. J., Mol. Biol., 166, 557-580, 1983); namely, a method in which the recombinant DNA is added to competent cells prepared in the presence of CaCl₂, MgCl₂, or RbC1. Further, as a vector other than a plasmid, a phage vector such as a lambda system may be used.

As a method for selecting a transformant containing the cDNA of interest from the resulting transformants, various methods such as (i) a method for screening a transformant using a synthetic oligonucleotide probe, (ii) a method for screening a transformant using a probe produced by PCR, (iii) a method for screening a transformant using an antibody against the polypeptide of the present invention, or (iv) a method for screening a transformant using a selective hybridization translation system, may be used.

In the method of the item (i) for screening a transformant using a synthetic oligonucleotide probe, the transformant containing the cDNA of interest may be selected, for example, by the following procedure.

An oligonucleotide which corresponds to the whole or a part of the polypeptide of the present invention is synthesized (in this case, it may be either a nucleotide sequence taking the codon usage into consideration or a plurality of nucleotide sequences as a combination of possible nucleotide sequences, and in the latter case, their numbers can be reduced by including inosine) and, using this oligonucleotide as a probe (labeled with ³²P or ³³P), hybridized with a nitrocellulose filter or a polyamide filter on which DNAs of the transformants are denatured and fixed, to screen and select resulting positive strains.

In the method of the item (ii) for screening a transformant using a probe produced by PCR, the transformant containing the cDNA of interest may be selected, for example, by the following procedure.

Oligonucleotides of a sense primer and an antisense primer corresponding to a part of the polypeptide of the present invention are synthesized, and a DNA fragment encoding the whole or a part of the polypeptide of interest is amplified by carrying out PCR using these primers in combination. As a template DNA used in this method, cDNA synthesized by a reverse transcription reaction from mRNA of cells capable of producing the polypeptide of the present invention, or genomic DNA, may be used. The resulting DNA fragment is labeled with ³²P or ³³P, and a transformant containing the cDNA of interest is selected by carrying out a colony hybridization or a plaque hybridization using this fragment as a probe.

In the method of the item (iii) for screening a transformant using an antibody against the polypeptide of the present invention, the transformant containing the cDNA of interest may be selected, for example, by the following procedure.

Polypeptides are produced into a culture supernatant, inside the cells, or on the cell surface of transformants. A transformant containing the cDNA of interest is selected by detecting a strain producing the desired polypeptide using an antibody against the polypeptide of the present invention and a second antibody against the first antibody.

In the method of the item (iv) for screening a transformant using a selective hybridization translation system, the transformant containing the cDNA of interest may be selected, for example, by the following procedure.

First, cDNA obtained from each transformant is blotted on, for example, a nitrocellulose filter and hybridized with mRNA prepared from cells capable of producing the polypeptide of the present invention, and then the mRNA bound to the cDNA is dissociated and recovered. The recovered mRNA is translated into a polypeptide in an appropriate polypeptide translation system, for example, injection into Xenopus oocytes or a cell-free system such as a rabbit reticulocyte lysate or a wheat germ. A transformant containing the cDNA of interest is selected by detecting it with the use of an antibody against the polypeptide of the present invention.

A method for collecting the polynucleotide of the present invention from the resulting transformant of interest can be carried out in accordance with a known method (for example, Sambrook, J. et al., “Molecular Cloning-A Laboratory Manual”, Cold Spring Harbor Laboratory, NY, 1989). For example, it may be carried out by separating a fraction corresponding to the plasmid DNA from cells and cutting out the cDNA region from the plasmid DNA.

In the chemical synthesis method of the item (3), the polynucleotide of the present invention may be produced, for example, by binding DNA fragments produced by a chemical synthesis method. Each DNA can be synthesized using a DNA synthesizer [for example, Oligo 1000M DNA Synthesizer (Beckman) or 394 DNA/RNA Synthesizer (Applied Biosystems)].

Further, the polynucleotide of the present invention may be produced by nucleic acid chemical synthesis in accordance with a conventional method such as a phosphite triester method (Hunkapiller, M. et al., Nature, 10, 105-111, 1984), based on the information on the polypeptide of the present invention. Tn this connection, codons for each amino acid are known and can be optionally selected and determined by the conventional method, for example, by taking a codon usage of each host to be used into consideration (Crantham, R. et al., Nucleic Acids Res., 9, r43-r74, 1981). Further, a partial modification of codons of these base sequences can be carried out in accordance with a conventional method, such as site directed mutagenesis which uses a primer comprised of a synthetic oligonucleotide coding for a desired modification (Mark, D. F. et al., Proc. Natl. Acad. Sci. USA, 81, 5662-5666, 1984).

Determination of the DNA sequences obtained by the above-mentioned methods can be carried out by, for example, a Maxam-Gilbert chemical modification method (Maxam, A. M. and Gilbert, W., “Methods in Enzymology”, 65, 499-559, 1980) or a dideoxynucleotide chain termination method (Messing, J. and Vieira, J., Gene, 19, 269-276, 1982).

An isolated polynucleotide of the present invention is re-integrated into an appropriate vector DNA and a eucaryotic or procaryotic host cell may be transfected by the resulting expression vector. Further, it is possible to express the polynucleotide in a desired host cell, by introducing an appropriate promoter and a sequence related to the gene expression into the vector.

The expression vector of the present invention is not particularly limited, so long as it comprises the polynucleotide of the present invention. As the expression vector, there may be mentioned, for example, an expression vector obtained by introducing the polynucleotide of the present invention into a known expression vector appropriately selected in accordance with a host cell to be used or a cell to be introduced. The expression vector of the present invention includes an expression vector for manufacturing the recombinant polypeptide of the present invention and an expression vector for producing the polypeptide of the present invention in a body by gene therapy.

The cell of the present invention is not particularly limited, so long as it is transfected with the expression vector of the present invention and comprises the polynucleotide of the present invention. The cell of the present invention may be, for example, a cell in which the polynucleotide is integrated into a chromosome of a host cell, or a cell containing the polynucleotide as an expression vector comprising polynucleotide. Further, the cell of the present invention may be a cell expressing the polypeptide of the present invention, or a cell not expressing the polypeptide of the present invention. The cell of the present invention may be obtained by, for example, transfecting a desired host cell with the expression vector of the present invention.

In the eucaryotic host cells, for example, cells of vertebrates, insects, and yeast are included. As the vertebral cell, there may be mentioned, for example, a simian COS cell (Gluzman, Y., Cell, 23, 175-182, 1981), a dihydrofolate reductase defective strain of a Chinese hamster ovary cell (CHO) (Urlaub, G. and Chasin, L. A., Proc. Natl. Acad. Sci. USA, 77, 4216-4220, 1980), a human fetal kidney derived HEK293 cell, a 293-EBNA cell (Invitrogen) obtained by introducing an EBNA-1 gene of Epstein Barr Virus into HEK293 cell, or a human 293T cell (DuBridge, R. B. et al., Mol. Cell. Biol., 7, 379-387, 1987).

As an expression vector for a vertebral cell, a vector containing a promoter positioned upstream of the gene to be expressed, an RNA splicing site, a polyadenylation site, a transcription termination sequence, and the like may be generally used. The vector may further contain a replication origin, if necessary. As the expression vector, there may be mentioned, for example, pSv2dhfr containing an SV40 early promoter (Subramani, S. et al., Mol. Cell. Biol., 1, 854-864, 1981), pEF-BOS containing a human elongation factor promoter (Mizushima, S. and Nagata, S., Nucleic Acids Res., 18, 5322, 1990), or pCEP4 containing a cytomegalovirus promoter (Invitrogen).

When the COS cell is used as the host cell, a vector which has an SV40 replication origin, can perform an autonomous replication in the COS cell, and has a transcription promoter, a transcription termination signal, and an RNA splicing site, may be used as the expression vector. As the vector, there may be mentioned, for example, pME18S (Maruyama, K. and Takebe, Y., Med. Immunol., 20, 27-32, 1990), pEF-BOS (Mizushima, S. and Nagata, S., Nucleic Acids Res., 18, 5322, 1990), or pCDM8 (Seed, B., Nature, 329, 840-842, 1987).

The expression vector may be incorporated into COS cells by, for example, a DEAE-dextran method (Luthman, H. and Magnusson, G., Nucleic Acids Res., 11, 1295-1308, 1983), a calcium phosphate-DNA co-precipitation method (Graham, F. L. and van der Ed, A. J., Virology, 52, 456-457, 1973), a method using a commercially available transfection reagent (for example, FuGENE™6 Transfection Reagent; Boeringer Mannheim), or an electroporation method (Neumann, E. et al., EMBO J., 1, 841-845, 1982).

When the CHO cell is used as the host cell, a transfected cell capable of stably producing the polypeptide of the present invention can be obtained by carrying out co-transfection of an expression vector comprising the polynucleotide encoding the polypeptide of the present invention, together with a vector capable of expressing a neo gene which functions as a G418 resistance marker, such as pRSVneo (Sambrook, J. et al., “Molecular Cloning-A Laboratory Manual”, Cold Spring Harbor Laboratory, NY, 1989) or pSV2-neo (Southern, P. J. and Berg, P., J. Mol. Appl. Genet., 1, 327-341,1982), and selecting a G418 resistant colony.

As a vector for a gene therapy, a vector generally used (for example, a retrovirus vector, an adenovirus vector, or Sendai virus vector) can be used.

The cell of the present invention may be cultured in accordance with the conventional method, and the polypeptide of the present invention is produced inside the cells. As a medium to be used in the culturing, a medium commonly used in a desired host cell may be appropriately selected. In the case of the COS cell, for example, a medium such as an RPMI-1640 medium or a Dulbecco's modified Eagle's minimum essential medium (DMEM) may be used, by supplementing it with a serum component such as fetal bovine serum (FBS) if necessary. In the case of the 293-EBNA cell, a medium such as a Dulbecco's modified Eagle's minimum essential medium (DMEM) with a serum component such as fetal bovine serum (FBS) and G418 may be used.

The polypeptide of the present invention produced inside the cell of the present invention by culturing the cells may be separated and purified therefrom by various known separation techniques making use of the physical properties, chemical properties and the like of the polypeptide. More particularly, the polypeptide of the present invention may be purified by treating a cell extract containing the polypeptide of the present invention with a commonly used treatment, for example, a treatment with a protein precipitant, ultrafiltration, various liquid chromatography techniques such as molecular sieve chromatography (gel filtration), adsorption chromatography, ion exchange chromatography, affinity chromatography, or high performance liquid chromatography (HPLC), or dialysis, or a combination thereof.

When the polypeptide of the present invention is expressed as a fusion protein with a marker sequence in frame, identification of the expression of the polypeptide of the present invention, purification thereof, or the like may be easily carried out. As the marker sequence, there may be mentioned, for example, a FLAG tag, a hexa-histidine tag, a hemagglutinin tag, or a myc epitope. Further, by inserting a specific amino acid sequence recognized by a protease such as enterokinase, factor Xa, or thrombin between the marker sequence and the polypeptide of the present invention, the marker sequence may be removed by the protease.

It is possible to screen a substance which modifies (for example, inhibits or promotes) an SMG-1 activity of the polypeptide according to the present invention, using the polypeptide of the present invention.

A substance inhibiting the SMG-1 activity of the polypeptide of the present invention (for example, an inhibitor of phosphatidyl inositol kinase related kinase, more particularly, for example, wortmannin or caffeine) can suppress NMD, and thus is useful as a candidate of an agent for treating and/or preventing a disease caused by at least a premature translation termination codon (PTC) generated by a nonsense mutation. The polypeptide of the present invention per se may be used as a screening tool for screening a substance inhibiting the SMG-1 activity of the polypeptide of the present invention, or for screening an agent for treating and/or preventing a disease caused by a nonsense mutation of a specific gene.

The term “nonsense mutation” as used herein includes, for example, a mutation in which a codon encoding an amino acid is changed to a termination codon, and a mutation in which a termination codon is generated by a point mutation or deletion at a splicing site, or by frame-shift caused by insertion and/or deletion of one or more bases.

The disease caused by one or more PTCs generated by a nonsense mutation is not particularly limited, but there may be mentioned, for example, a genetic disease (for example, Duchenne type muscular dystrophy), cancer due to a somatic mutation, or the like. The important point is that, among all diseases due to genome mutation, almost all diseases “due to one or more PTCs by a nonsense mutation” are included in such diseases.

One-quarter of the diseases due to genome mutations have the termination codon in the middle of a specific gene. The reasons for these diseases are that the protein consisting of the full-length polypeptide inherently encoded by the gene is not expressed, and that, due to the presence of the NMD mechanism, almost no protein fragments consisting of the N terminal side partial fragments of the full length polypeptide inherently encoded by the gene are expressed. However, even if there is a termination codon in the middle of the gene, and even if in the state of a protein fragment, there are not a few cases of activity of the same extent as that of full length polypeptide or the minimum necessary level, depending on the type of the gene or the position of the termination codon. In this case, if it were possible to inhibit the NMD mechanism, it would become possible to express a protein fragment having an effective activity, and thus it is theoretically predicted that at least part of a disease due to the presence of a termination codon in the middle of a specific gene, that is, a disease due to nonsense mutation of a specific gene can be alleviated. However, no technique for a specific suppression of NMD has been known at all in the past.

Among the substances selected by the screening method of the present invention, a substance inhibiting the SMG-1 activity of the polypeptide of the present invention can specifically suppress NMD through inhibition of the SMG-1 activity of the polypeptide of the present invention, and thus is useful as an active ingredient of a new type of agent for treatment and/or prevention which can alleviate gene mutations for at least part of all sorts of diseases due to the nonsense mutation of specific genes.

On the other hand, among the substances selected by the screening method of the present invention, a substance promoting the SMG-1 activity of the polypeptide of the present invention can promote NMD, and thus is useful as an active ingredient of an agent for promoting NMD or an agent for treating and/or preventing a disease due to mRNA having one or more PTCs, which should be removed, not being removed.

As the screening method of the present invention, there may be mentioned, on the basis of differences in methods for evaluating the degree of an SMG-1 activity,

(A) a method comprising the steps of: bringing into contact (1) the polypeptide of the present invention, (2) Upf1/SMG-2 (for example, human Upf1/SMG-2), a fragment thereof capable of being phosphorylated, or a fusion polypeptide comprising Upf1/SMG-2 or the fragment thereof, and (3) a substance to be tested, and carrying out phosphorylation under the conditions that the polypeptide of the present invention is brought into contact with Upf1/SMG-2, the fragment thereof, or the fusion polypeptide, and analyzing whether or not Upf1/SMG-2, the fragment thereof, or the fusion polypeptide is phosphorylated (hereinafter referred to as a Upf1/SMG-2 type screening method); or

(B) a method comprising the steps of: bringing (1) the polypeptide of the present invention into contact with (2) a substance to be tested, and carrying out phosphorylation under the conditions that the polypeptide is brought into contact with the substance to be tested, and analyzing whether or not the polypeptide is autophosphorylated (hereinafter referred to as a autophosphorylation type screening method).

Substances to be tested which may be applied to the detection method or screening method of the present invention are not particularly limited, but there may be mentioned, for example, various known compounds (including peptides) registered in chemical files, compounds obtained by combinatorial chemistry techniques (Terrett, N. K. et al., Tetrahedron, 51, 8135-8137, 1995) or conventional synthesis techniques, or random peptides prepared by employing a phage display method (Felici, F. et al., J. Mol. Biol., 222, 301-310, 1991) or the like. In addition, culture supernatants of microorganisms, natural components derived from plants or marine organisms, or animal tissue extracts may be used as the test Substances for screening. Further, compounds (including peptides) obtained by chemically or biologically modifying compounds (including peptides) selected by the screening method of the present invention may be used.

The Upf1/SMG-2 type screening method of the present invention (hereinafter, the explanation will be given of a Upf1/SMG-2 type screening method of the present invention using as an example the case of the use of Upf1/SMG-2 as the “Upf1/SMG-2, a fragment thereof capable of being phosphorylated, or a fusion polypeptide comprising the same”) can be performed in the same way as the above-mentioned method of judgment of the SMG-1 activity, except that, instead of bringing the test polypeptide into contact with Upf1/SMG-2, the polypeptide of the present invention, Upf1/SMG-2, and the test substance are brought into contact. That is, it is possible to judge whether or not the test substance modifies (for example, inhibits or promotes) the SMG-1 activity of the polypeptide of the present invention, by bringing into contact the polypeptide of the present invention, Upf1/SMG-2, and the test substance, carrying out phosphorylation in the presence of the test substance, and then analyzing whether or not Upf1/SMG-2 is phosphorylated. For example, if the Upf1/SMG-2 is not phosphorylated or the degree of the phosphorylation thereof decreases in the presence of the test substance, it is possible to judge that the test substance is a substance inhibiting the SMG-1 activity of the polypeptide of the present invention. On the other hand, if the degree of the phosphorylation of Upf1/SMG-2 increases in the presence of the test substance, compared with the case of the absence of the test substance, it is possible to judge that the test substance is a substance promoting the SMG-1 activity of the polypeptide of the present invention.

The SMG-1 activity may be judged by not only the presence or degree of phosphorylation of Upf1/SMG-2, but also the presence or degree of the autophosphorylation of the polypeptide per se of the present invention, as shown, for example, in Example 6(2), Example 7(3), or Example 9(1).

With the autophosphorylation type screening method of the present invention, it is possible to judge whether or not the test substance modifies (for example, inhibits or promotes) the SMG-1 activity of the polypeptide of the present invention, by bringing the polypeptide of the present invention into contact with the test substance, carrying out phosphorylation in the presence of the test substance, and then analyzing whether or not the polypeptide is autophosphorylated. For example, when the polypeptide of the present invention is not phosphorylated or the extent of the phosphorylation thereof decreases in the presence of the test substance, it is possible to judge that the test substance is a substance inhibiting the SMG-1 activity of the polypeptide of the present invention. On the other hand, when the extent of phosphorylation of the polypeptide of the present invention increases in the presence of the test substance, compared with the case of the absence of the test substance, it is possible to judge that the test substance is a substance promoting the SMG-1 activity of the polypeptide of the present invention.

The substance inhibiting the SMG-1 activity (for example, an inhibitor of phosphatidyl inositol kinase related kinase inhibitor, more particularly, for example, wortmannin or caffeine), which may be selected by the screening method of the present invention, can inhibit NMD and is useful as a candidate for an agent for treating and/or preventing a disease caused by one or more PTCs generated by a nonsense mutation. The substance inhibiting an SMG-1 activity (hereinafter sometimes referred to as an SMG-1 inhibitor) can be administered to a subject (for example, an animal, preferably a mammal, particularly a human) in need of suppressing NMD or in need of treating and/or preventing a disease caused by one or more PTCs generated by a nonsense mutation, with or without, but preferably with, a pharmaceutically or veterinarilv acceptable ordinary carrier or diluent, in an amount effective therefor.

The agent of the present invention for suppressing NMD or for treating and/or preventing a disease caused by one or more PTCs generated by a nonsense mutation comprises the SMG-1 inhibitor (preferably an inhibitor of a phosphatidyl inositol kinase related kinase, more preferably wortmannin or caffeine) as an active ingredient, and may further comprise a pharmaceutically or veterinarily acceptable ordinary carrier or diluent.

The pharmaceutical composition of the present invention for suppressing NMD or for treating and/or preventing a disease caused by one or more PTCs generated by a nonsense mutation comprises the SMG-1 inhibitor (preferably an inhibitor of a phosphatidyl inositol kinase related kinase, more preferably wortmannin or caffeine) as an active ingredient, and a pharmaceutically or veterinarily acceptable ordinary carrier or diluent.

As one method for the treatment of a disease caused by one or more PTCs generated by a nonsense mutation, experiments into the use of “nonsense suppression” have been made. “Nonsense suppression” means the phenomenon of the production of a full-length protein by a read through of one or more PTCs, even with a gene having the PTCs. It is known that several aminoglycoside antibiotics induce nonsense suppression. Experiments are beginning on utilizing the aminoglycoside antibiotics for serious genetic diseases such as cystic fibrosis or muscular dystrophy, to alleviate symptoms [Clancy, J. P. et al., Am. J. Respir. Crit. Care Med., 163 (7), 1683-1692 (2001) or Wagner, K. R. et al., Ann. Neurol., 49 (6), 706-711 (2001)]. However, mRNA transcribed from a gene having one or more PTCs is removed by NMD, and thus the amount of the mRNA is small and the effect is not sufficient.

The substance inhibiting the SMG-1 activity (for example, an inhibitor of phosphatidyl inositol kinase related kinase inhibitor, more particularly, for example, wortmannin or caffeine), which may be selected by the screening method of the present invention has an activity of suppressing NMD. Therefore, when using such an SMG-1 inhibiting substance together with the aminoglycoside antibiotic, even with a gene having one or more PTCs, a suppression of NMD enables an increase of the amount of mRNA and enables the efficient production of a full-length protein by a read through of the PTCs.

Further, as shown in Example 8(2) or Example 8(3), when overexpressing an SMG-1-activity-deficient mutant [for example, 6H-hSMG-1(DA) used in Example 8(3)] in cells, compared with the case of the absence of an SMG-1-activity-deficient mutant, the amount of mRNA derived from the gene having one or more PTCs increases. Further, as shown in Example 13(1) and Example 13(2), when adding an SMG-1 inhibitor to cells, the amount of mRNA derived from the gene having one or more PTCs increases. Therefore, by using the SMG-1 inhibitor or SMG-1-activity-deficient mutant together with the aminoglycoside antibiotic, even with a gene having one or more PTCs, it is possible to increase the amount of mRNA by a suppression of NMD, and possible to efficiently produce a full-length protein by a read through of the PTCs.

As above, the SMG-1 inhibitor or the SMG-1-acitivity-deficient mutant and the aminoglycoside antibiotic can be administered to a subject (for example, an animal, preferably a mammal, particularly a human) in need of suppressing nonsense, with or without, but preferably with, a pharmaceutically or veterinarily acceptable ordinary carrier or diluent in an amount effective therefor, to effectively produce a full-length protein on the basis of nonsense suppression.

The agent of the present invention for suppressing nonsense comprises the SMG-1 inhibitor (preferably an inhibitor of a phosphatidyl inositol kinase related kinase, more preferably wortmannin or caffeine) or the SMG-1-acitivity-deficient mutant as an active ingredient, and the aminoglycoside antibiotic as an active ingredient, and may further comprise a pharmaceutically or veterinarily acceptable ordinary carrier or diluent.

The pharmaceutical composition of the present invention for suppressing nonsense comprises the SMG-1 inhibitor (preferably an inhibitor of a phosphatidyl inositol kinase related kinase, more preferably wortmannin or caffeine) or the SMG-1-acitivity-deficient mutant as an active ingredient, the aminoglycoside antibiotic as an active ingredient, and a pharmaceutically or veterinarily acceptable ordinary carrier or diluent.

The aminoglycoside antibiotic which may be used in the pharmaceutical composition of the present invention for suppressing nonsense is not particularly limited, so long as it has a nonsense suppression activity alone. As the aminoglycoside antibiotic, there may be mentioned, for example, gentamycin or G418.

The SMG-1-acitivity-deficient mutant which may be used in the pharmaceutical composition of the present invention for suppressing nonsense is not particularly limited, so long as it does not exhibit the SMG-1 activity (i.e., activity of phosphorylating Upf1/SMG-2) and comprising an amino acid sequence in which one or plural amino acids are deleted, substituted, and/or inserted at one or plural positions in the amino acid sequence consisting of 129th to 3657th amino acids in the amino acid sequence of SEQ ID NO: 2, and further, when overexpressed in cells having a gene with one or more PTCs, increases the amount of mRNA derived from the gene, compared with the case in the absence thereof. As the SMG-1-acitivity-deficient mutant, there may be mentioned, for example, a polypeptide wherein asparatic acid corresponding to the 2331st asparatic acid (D) in the amino acid sequence of SEQ ID NO: 2 is replaced with alanine (A).

Regarding nonsense suppression, it is known that three types of upf genes relate to an efficient termination of translation in yeast, and that any mutation of these genes will cause nonsense suppression [Wang, W. et al., EMBO J., 20 (4), 880-890 (2001)]. Further, from several observations in C. elegans, it is known that the ratio of nonsense suppression rises in smg-gene-deficient mutants [Page, M. F. et al., Mol. Cell. Biol., 19, 5943-5951 (1999)]. These known facts in yeast or C. elegans show that the smg gene is important in guaranteeing the strictness of termination of translation. In mammals, it is considered that SMG/UPF proteins (including human SMG-1) have similar functions. Therefore, the SMG-1 inhibitor, which may be selected by the screening method of the present invention, alone (that is, without the use together of the aminoglycoside antibiotic) can increase the amount of mRNA by NMD suppression, even with a gene having one or more PTCs, and thus efficiently produce a full-length protein by a read through of the PTCs. In this case, the SMG-1 inhibitor can efficiently produce the full-length protein by two types of different mechanisms (that is, NMD inhibition increasing the mRNA level, and suppression of termination of translation leading to synthesis of a full-length protein by a read through).

As described above, the SMG-1 inhibitor may be administered alone, or preferably, together with a pharmaceutically or veterinarily acceptable ordinary carrier or diluent, to a subject (for example, an animal, preferably a mammal, particularly a human) in need of nonsense suppression in an effective dosage, so as to efficiently produce a full-length protein on the basis of nonsense suppression.

The agent for nonsense suppression of the present invention comprises the SMG-1 inhibitor (preferably an inhibitor of a phosphatidyl inositol kinase related kinase, more preferably wortmannin or caffeine) as an active ingredient, and may further comprise a pharmaceutically or veterinarily acceptable ordinary carrier or diluent.

The pharmaceutical composition of the present invention for nonsense suppression comprises the SMG-1 inhibitor (preferably an inhibitor of a phosphatidyl inositol kinase related kinase, more preferably wortmannin or caffeine) as an active ingredient, and a pharmaceutically or veterinarily acceptable ordinary carrier or diluent.

The substance promoting the SMG-1 activity (hereinafter sometimes referred to simply as “substance promoting SMG-1”) which may be selected by the screening method of the present invention can promote NMD, and is useful as a candidate of an agent for treating and/or preventing a disease caused by mRNA having one or more PTCs, which should be removed, not being removed. Further, as shown in Example 8(2) or Example 8(3), when introducing a polynucleotide encoding the polypeptide of the present invention [for example, 6H-hSMG-1 used in Example 8(3)] into cells and overexpressing the polypeptide in the cells, NMD can be promoted (that is, the amount of mRNA derived from a gene having one or more PTCs can be further reduced).

As described above, the substance promoting SMG-1, the polypeptide of the present invention, or the polynucleotide encoding the polypeptide may be administered alone, or preferably, together with a pharmaceutically or veterinarily acceptable ordinary carrier or diluent, to a subject (for example, an animal, preferably a mammal, particularly a human) in need of promoting NMD, or in need of treating and/or preventing a disease caused by mRNA having one or more PTCs, which should be removed, not being removed, in an effective dosage.

The agent for promoting NMD of the present invention comprises the substance promoting SMG-1, the polypeptide of the present invention, or the polynucleotide encoding the polypeptide as an active ingredient, and may further comprise a pharmaceutically or veterinarily acceptable ordinary carrier or diluent.

The pharmaceutical composition of the present invention for promoting NMD comprises the substance promoting SMG-1, the polypeptide of the present invention, or the polynucleotide encoding the polypeptide as an active ingredient, and a pharmaceutically or veterinarily acceptable ordinary carrier or diluent.

The formulation of the pharmaceutical composition of the present invention is not particularly limited to, but may be, for example, oral medicines, such as powders, fine particles, granules, tablets, capsules, suspensions, emulsions, syrups, extracts or pills, or parenteral medicines, such as injections, liquids for external use, ointments, suppositories, creams for topical application, or eye lotions.

The oral medicines may be prepared by an ordinary method using, for example, fillers, binders, disintegrating agents, surfactants, lubricants, flowability-enhancers, diluting agents, preservatives, coloring agents, perfumes, tasting agents, stabilizers, humectants, antiseptics, antioxidants or the like, such as gelatin, sodium alginate, starch, corn starch, saccharose, lactose, glucose, mannitol, carboxylmethylcellulose, dextrin, polyvinyl pyrrolidone, crystalline cellulose, soybean lecithin, sucrose, fatty acid esters, talc, magnesium stearate, polyethylene glycol, magnesium silicate, silicic anhydride, or synthetic aluminum silicate.

The parenteral administration may be, for example, an injection such as a subcutaneous or intravenous injection, or a per rectum administration. Of the parenteral formulations, an injection is preferably used.

When the injections are prepared, for example, water-soluble solvents, such as physiological saline or Ringer's solution, water-insoluble solvents, such as plant oil or fatty acid ester, agents for rendering isotonic, such as glucose or sodium chloride, solubilizing agents, stabilizing agents, antiseptics, suspending agents, or emulsifying agents may be optionally used, in addition to the active ingredient.

The pharmaceutical composition of the present invention may be administered in the form of a sustained release preparation using sustained release polymers. For example, the pharmaceutical composition of the present invention may be incorporated to a pellet made of ethylenevinyl acetate polymers, and the pellet may be surgically implanted in a tissue to be treated.

The pharmaceutical composition of the present invention may contain the active ingredient in an amount of, but is by no means limited to, 0.01 to 99% by weight, preferably 0.1 to 80% by weight.

A dose of the pharmaceutical composition of the present invention is not particularly limited, but may be determined dependent upon, for example, the kind of the active ingredient, the kind of disease, the age, sex, body weight, or symptoms of the subject, a method of administration, or the like. The pharmaceutical composition of the present invention may be orally or parenterally administered.

The pharmaceutical composition of the present invention may be administered as a medicament or in various forms, for example, eatable or drinkable products, such as functional foods or health foods, or feeds.

An antibody, such as a polyclonal antibody or a monoclonal antibody, which reacts with the polypeptide of the present invention may be obtained by directly administering the polypeptide of the present invention or a fragment thereof to various animals. Alternatively, it may be obtained by a DNA vaccine method (Raz, E. et al., Proc. Natl. Acad. Sci. USA, 91, 9519-9523, 1994; or Donnelly, J. J. et al., J. Infect. Dis., 173, 314-320, 1996), using a plasmid into which a polynucleotide encoding the polypeptide of the present invention is inserted.

The polyclonal antibody may be produced from a serum or eggs of an animal such as a rabbit, a rat, a goat, or a chicken, in which the animal is immunized and sensitized by the polypeptide of the present invention or a fragment thereof emulsified in an appropriate adjuvant (for example, Freund's complete adjuvant) by intraperitoneal, subcutaneous, or intravenous administration. The polyclonal antibody may be separated and purified from the resulting serum or eggs in accordance with conventional methods for polypeptide isolation and purification. Examples of the separation and purification methods include, for example, centrifugal separation, dialysis, salting-out with ammonium sulfate, or a chromatographic technique using such as DEAE-cellulose, hydroxyapatite, protein A agarose, and the like.

The monoclonal antibody may be easily produced by those skilled in the art, according to, for example, a cell fusion method of Kohler and Milstein (Kohler, G. and Milstein, C., Nature, 256, 495-497, 1975).

A mouse is immunized intraperitoneally, subcutaneously, or intravenously several times at an interval of a few weeks by a repeated inoculation of emulsions in which the polypeptide of the present invention or a fragment thereof is emulsified into a suitable adjuvant such as Freund's complete adjuvant. Spleen cells are removed after the final immunization, and then fused with myeloma cells to prepare hybridomas.

As a myeloma cell for obtaining a hybridoma, a myeloma cell having a marker such as a deficiency in hypoxanthine-guanine phosphoribosyltransferase or thymidine kinase (for example, mouse myeloma cell line P3X63Ag8.U1) may be used. As a fusing agent, polyethylene glycol may be used. As a medium for preparation of hybridomas, for example, a commonly used medium such as an Eagle's minimum essential medium, a Dulbecco's modified minimum essential medium, or an RPMI-1640 medium may be used by adding properly 10 to 30% of a fetal bovine serum. The fused strains may be selected by a HAT selection method. A culture supernatant of the hybridomas is screened by a well-known method such as an ELISA method or an immunohistological method, to select hybridoma clones secreting the antibody of interest. The monoclonality of the selected hybridoma is guaranteed by repeating subcloning by a limiting dilution method. Antibodies in an amount which may be purified are produced by culturing the resulting hybridomas in a medium for 2 to 4 days, or in the peritoneal cavity of a pristane-pretreated BALB/c strain mouse for 10 to 20 days.

The resulting monoclonal antibodies in the culture supernatant or the ascites may be separated and purified by conventional polypeptide isolation and purification methods. Examples of the separation and purification methods include, for example, centrifugal separation, dialysis, salting-out with ammonium sulfate, or chromatographic technique using such as DEAE-cellulose, hydroxyapatite, protein A agarose, and the like.

Further, the monoclonal antibodies or the antibody fragments containing a part thereof may be produced by inserting the whole or a part of a gene encoding the monoclonal antibody into an expression vector and introducing the resulting expression vector into appropriate host cells (such as E. coli, yeast, or animal cells) Antibody fragments comprising an active part of the antibody such as F(ab′)₂, Fab, Fab′, or Fv may be obtained by a conventional method, for example, by digesting the separated and purified antibodies (including polyclonal antibodies and monoclonal antibodies) with a protease such as pepsin or papain, and separating and purifying the resulting fragments by standard polypeptide isolation and purification methods.

Further, an antibody which reacts to the polypeptide of the present invention may be obtained in a form of single chain Fv or Fab in accordance with a method of Clackson et al. or a method of Zebedee et al. (Clackson, T. et al., Nature, 352, 624-628, 1991; or Zebedee, S. et al., Proc. Natl. Acad. Sci. USA, 89, 3175-3179, 1992). Furthermore, a humanized antibody may be obtained by immunizing a transgenic mouse in which mouse antibody genes are substituted with human antibody genes (Lonberg, N. et al., Nature, 368, 856-859, 1994).

The knock-out non-human animal of the present invention is not particularly limited, so long as the expression of the gene encoding the polypeptide of the present invention is partially or completely suppressed. It may be prepared by a method known per se.

For example, by using a recombinant vector containing the polynucleotide of the present invention, and embryonic stem cells of the target non-human animal, such as cow, sheep, goat, pig, horse, mouse, or chicken, the gene encoding the polypeptide of the present invention on the chromosomes thereof is deactivated by a known homologous recombinant technique [for example, Nature, 326, 6110, 295 (1987); or Cell, 51, 3, 503 (1987)], or is replaced with any sequence [for example, Nature, 350, 6315, 243 (1991)], to prepare mutant clones. By using the mutant clones of the embryonic stem cells, a chimeric individual consisting of embryonic stem cell clones and normal cells may be prepared by a technique such as an aggregation chimera method or an injection chimera method into blastocyst in fertilized eggs of the animal. By combining the chimera individual and a normal individual, it is possible to obtain an individual having any mutation in the gene encoding the polypeptide of the present invention located on the chromosomes of cells of the entire body. By further combining the individuals, it is possible to obtain, from homozygous individuals with the mutation in both homologous chromosomes, a knock-out non-human animal as an individual in which the expression of the gene encoding the polypeptide of the present invention is partially or completely suppressed.

Further, by introducing mutation into any site of a gene encoding the polypeptide of the present invention on the chromosome, it is also possible to produce a knock-out non-human animal. For example, by substituting, deleting, and/or inserting one or more bases with respect to a translation region of the gene encoding the polypeptide of the present invention on the chromosome, it is possible to modify the activity of the gene product.

Further, by introducing a similar mutation in the expression control region, it is also possible to modify, for example, the degree of expression, period of expression, and/or a tissue specificity. Further, by the combination with the Cre-loxP system, it is possible to control, for example, the period of expression, location of expression, and/or the amount of expression more directly. As such examples, an example in which, using a promoter expressed at a specific region of the brain, the target gene was deleted at only the specific region [Cell, 87, 7, 1317, 1996)], or an example in which, using an adenovirus expressing Cre, the target gene was deleted from the specific organ at the desired period [Science, 278, 5335 (1997)] are known.

Therefore, even for a gene encoding the polypeptide of the present invention on the chromosome, it is possible to control expression at any period or tissue as described above. Further, it is possible to prepare a knock-out non-human animal having any insertion, deletion, and/or substitution at the translation region or expression control region. A knock-out non-human animal can induce the symptoms of various diseases derived from the polypeptide of the present invention at any period, to any degree, and/or at any location. As described above, the knock-out non-human animal of the present invention becomes an extremely useful animal model in the treatment or prevention of various diseases derived from the polypeptide of the present invention.

Further, the knock-out non-human animal of the present invention can be used to establish a model animal of a disease due to a gene different from the gene encoding the polypeptide of the present invention. For example, one of the knock-out non-human animals of the present invention, that is, an SMG-1 knock-out mouse, and various lines of (apparent) normal mice can be combined. When the normal mouse contains a mutant gene having one or more PTCs, in a mouse obtained by the combination (for example, a homozygous individual having the SMG-1 mutation in both of the homologous chromosomes or a heterozygous individual having the SMG-1 mutation in one of the homologous chromosomes), NMD is suppressed, and thus the mRNA derived from the mutant gene increases. As a result, for a certain mutant gene, sometimes hidden symptoms surface and some sort of disease appears. It is possible to establish a new disease model mouse.

The method of the present invention for identifying a nonsense mutation point in a gene having a nonsense mutation by one or more PTCs is not particularly limited, so long as it comprises a step of culturing a cell to be tested which is obtained from a subject to be tested and may contain a gene having a nonsense mutation by one or more PTCS, in the presence of an inhibitor of an SMG-1 activity; and a step of analyzing molecular weight of a polypeptide derived from the gene in the cultured cell. The method can be performed, for example, in accordance with the method shown in Example 13(2).

According to the nonsense mutation point identification method of the present invention, in a gene which may have a nonsense mutation by one or more PTCs or a gene which is known to have a nonsense mutation by one or more PTCs, it is possible to identify at what site the nonsense mutation by the PTCs is located.

At the culturing step in the nonsense mutation point identification method of the present invention, test cells which may have one or more genes having a nonsense mutation by one or more PTCs, taken from the examined subject, are cultured in the presence of an SMG-1 inhibitor (for example, phosphatidyl inositol kinase related kinase inhibitor, more particularly, for example, wortmannin or caffeine). For example, in Example 13(2), two types of cultured cells having a PTC in the p53 gene were used as test cells. More particularly, the lung adenocarcinoma cell line Calu6 containing the PTC in the 196th codon of the p53 gene (number of amino acid residues=393) and small cell lung carcinoma cell line N417 containing the PTC in the 298th codon of the p53 gene were used.

With the nonsense mutation point identification method of the present invention, as a control, it is preferable to use cells containing normal genes without PTCs. For example, in Example 13(2), the cultured cell A549 having the normal p53 gene without PTCs was used. In this connection, when not using such a control, for example, it is necessary to determine in advance the molecular weight of the polypeptide derived from the normal gene without PTCs.

At the analyzing step in the nonsense mutation point identification method of the present invention, the molecular weight of the polypeptide derived from the gene to be analyzed in the test cells obtained at the culturing step is analyzed. As the method of analysis of the molecular weight of the polypeptide, a known analysis method, for example, the Western blotting, may be mentioned. For example, in Example 13(2), with the cultured cell N417 containing the PTC at the 298th codon of the p53 gene, the molecular weight of the polypeptide derived from the gene was approximately 40 kDa, while with the cultured cell A549 (control) having the normal p53 gene without PTCs, the molecular weight of the polypeptide derived from the gene was approximately 53 kDa, as shown in FIG. 31. Therefore, from the comparison of these molecular weights, it is possible to determine that there is a PTC at the approximately 40/53 position from the 5′ terminal of the p53 gene. This value matches with the fact that the 298th codon is the PTC in all 393 codons.

The method of the present invention for detecting a gene having a nonsense mutation is not particularly limited, so long as it comprises a step of culturing at least two groups of cells to be tested, which are obtained from a subject to be tested and may contain a gene having a nonsense mutation by one or more PTCs, in the presence of an inhibitor of an SMG-1 activity and in the absence thereof, respectively; and a step of detecting a presence or absence of the difference in an amount of mRNA derived from the gene in the cultured cells. The method may be performed, for example, in accordance with the method shown in Example 13(2).

According to the method of the present invention for detecting a gene having a nonsense mutation, even with a gene in which the presence or absence of PTCs is not known at all, it is possible to detect whether or not the gene has a nonsense mutation by one or more PTCs.

At the culturing step in the method of the present invention for detecting a gene having a nonsense mutation, at least two groups of test cells which may contain a gene having a nonsense mutation by one or more PTCs, taken from the examined subject, are cultured in the presence and absence of an SMG-1 inhibitor (for example, phosphatidyl inositol kinase related kinase inhibitor, more particularly, for example, wortmannin or caffeine), respectively. For example, in Example 13(2), two types of cultured cells having a PTC in the p53 gene were used as the test cells. More particularly, the lung adenocarcinoma cell line Calu6 containing PTC in the 196th codon of the p53 gene (number of amino acid residues=393) and the small cell lung adenocarcinoma cell line N417 containing PTC in the 298th codon of the p53 gene were used. In this connection, in Example 13(2), the cultured cell A549 having the normal p53 gene without PTCs was used for a comparison.

At the step of analysis in the method of the present invention for detecting a gene having a nonsense mutation, any difference in the amount of mRNA derived in the gene in the test cells obtained at the culturing step is detected. As the method of detection of a difference in the amount of mRNA, a known analysis method, for example, the Northern blotting, may be mentioned. For example, in Example 13(2), with the cultured cell N417 containing the PCT at the 298th codon of the p53 gene, the amount of mRNA was reduced in the absence of the SMG-1 inhibitor, compared with the case of the presence of the SMG-1 inhibitor, and a difference occurred in the amount of mRNA, as shown in FIG. 31 or FIG. 32. On the other hand, as shown in FIG. 31, with cultured cell A549 having the normal p53 gene without PTCs (for comparison), the amount of mRNA did not change even in the absence of the SMG-1 inhibitor, compared with the case of the presence of the SMG-1 inhibitor, and no difference occurred in the amount of mRNA.

As described above, when the amount of mRNA is reduced in the absence of the SMG-1 inhibitor, compared with the case of the presence of the SMG-1 inhibitor, and a difference occurs in the amount of mRNA, it is possible to judge that the gene has a nonsense mutation by one or more PTCs. On the other hand, when no difference occurs in the amount of mRNA in the presence and absence of the SMG-1 inhibitor, it can be judged that the gene does not have a nonsense mutation by the PTCs.

### EXAMPLES

The present invention now will be further illustrated by, but is by no means limited to, the following Examples.

### Example 1

### Cloning of Human SMG-1 (hSMG-1) cDNA

The present inventor discovered that the N-terminus of the amino acid sequence encoded by the human cDNA clone KIAA0421 [Ishikawa, K. et al., DNA Res., 4, 307 (1997); GenBank access no. AB007881] has homology with the amino acid sequence characteristic of the kinase domain conserved in the PIKK family, and that the C-terminus has homology with the amino acid sequence characteristic of the FAT domain conserved in the PIKK family [Bosotti et al., Trends Biochem. Sci., 25, 225 (2000)]. Therefore, the human cDNA clone KIAA0421 was considered to be a novel cDNA of the PIKK family, but while this base sequence includes a termination codon and 3′ nontranslation region, there is no sequence capable of being specified as the start codon, and thus it was considered that the cDNA was of incomplete length. Therefore, to clarify the base sequence of the full-length cDNA, it was attempted to obtain the further 5′ side cDNA clone from the clone KIAA0421.

Using a cDNA fragment of the human cDNA clone KIAA0421 as a probe, a clone C was isolated from a cDNA library of the human cell line HeLa (Clonetech). Similarly, a clone yama9 (Y9) was isolated from a HeLa cDNA library [Chambon et al., Proc. Natl. Acad. Sci. USA, 86 (14), 5310-5314], a clone liver33 (Liv33) was isolated from a human liver library (Clonetech), and a clone muscle29 (mus29) was isolated from a human muscle library (Clonetech). Further, other various clones were isolated. The base sequences thereof were determined.

Next, a combination of a forward primer consisting of the base sequence of SEQ ID NO: 3 and a reverse primer consisting of the base sequence of SEQ ID NO: 4 was used to obtain a clone gap1 by a reverse transcription polymerase chain reaction (RT-PCR) method using the Total RNA of the human cell line HeLa. The RT-PCR was performed by using a commercially available kit (Ready-To-Go RT-PCR beads; Pharmacia), and performing an RT reaction at 42° C. for 30 minutes, then performing heat denaturation at 95° C. (3 minutes), repeating a cycle of 95° C. (1 minute), 54° C. (1 minute), and 72° C. (1 minute) 32 times, and finally performing an elongation reaction at 72° C. (7 minutes).

Further, a combination of a forward primer consisting of the base sequence of SEQ ID NO: 5 and a reverse primer consisting of the base sequence of SEQ ID NO: 6 was used to obtain a clone gap2 by the RT-PCR method using the Total RNA of the human cell line HeLa. The RT-PCR was performed under, the same conditions as the RT-PCR when obtaining the clone gap1.

It was attempted to connect the base sequences of these clones, but there was no sequence capable of being specified as the start codon, and only a base sequence of cDNA of an incomplete length could be obtained.

Therefore, a search for an EST having a sequence matching with the obtained base sequence was made in the base sequence database (GenBank), whereupon the human EST clone AI005513 (Research Genetics) was found. The base sequence of this EST has a start codon ATG in its frame, so the EST of the region including the start coden of the full-length cDNA consisting of the human cDNA clone KIAA0421 and its upstream region was estimated.

By determining the base sequence of the human EST clone AI005513, the base sequence of the cDNA consisting of the human cDNA clone KIAA0421 and its upstream region was clarified. The base sequence was that of SEQ ID NO: 1. When the base sequence database (GenBank) was searched, it was found that this base sequence was novel.

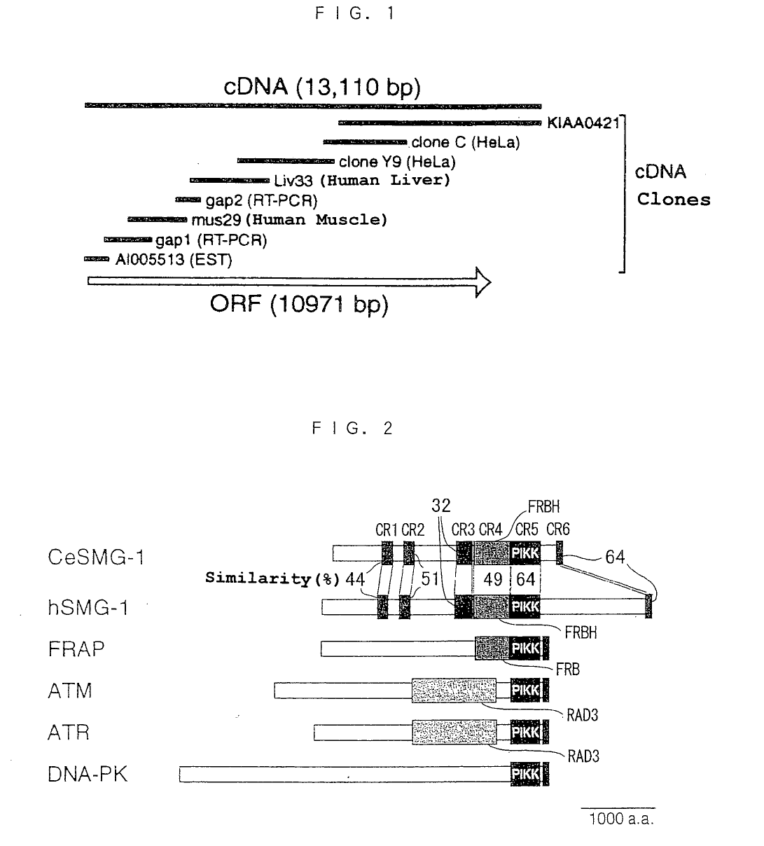

The relationship between the obtained cDNA clones and the novel base sequences and open reading frame (ORF) obtained therefrom is shown in FIG. 1. The length of the cDNA consisting of KIAA0421 and its upstream region, obtained from each cDNA clone, was approximately 13 kb. There was an approximately 11 kb open reading frame (ORF) encoding a protein consisting of 3657 amino acids. The estimated molecular weight of the protein encoded by the ORF was approximately 430 kDa, which matched the roughly calculated molecular weight of the endogenous molecule (p430) detected in Example 5(1).

A search of homology was conducted for the amino acid sequence (amino acid sequence of SEQ ID NO: 2) encoded by the ORF, whereupon it was found that there was a homology with the PIKK family FRAP (FKBP12-rapamycin associated protein)/mTOR (mammalian target of rapamycin)/RAFT1 (rapamycin and FKBP-target 1), ATM (ataxia telangiectasia mutated), ATR (ATM- and Rad3-related)/FRAPI, DNA-PKcs (DNA-PK catalytic subunit) and the like. The results of a comparison of human SMG-1 and known proteins are shown in FIG. 2.

In FIG. 2, the deduced PIKK related domain is shown by the black square. The FKBP12/rapamycin binding region (FRB) and its homologous region (FRBH) is shown by the dark gray, and the RAD3 homologous region is shown by the light gray. CR1 to CR6 mean regions with a high homology with C. elegans SMG1 (CeSMG1), and “1000 a.a.” shows the length of 1000 amino acid residues. Further, the numerical values of the homology are from GeneWorks ver 2 5.1 (IntelliGenetics). GenBank access number of FRAP is L34075, that of ATM is U33841, that of ATR is U76308i and that of DNA-PKcs is U34994.

In human SMG-1, the CR1 is the region consisting of the 557th to 727th amino acids. Similarly, the CR2 is the region consisting of the 911st to 1051st amino acids, the CR3 is the region consisting of the 1560th to 1756th amino acids, the CR4 is the region consisting of the 1785th to 2107th amino acids, the CR5 is the region consisting of the 2141st to 2422nd amino acids, and the CR6 is the region consisting of the 3602nd to 3657th amino acids.

Further, the region consisting of the 2130th to 2136th amino acids in the human SMG-1 is an amino acid sequence capable of functioning as an NLS (nuclear localization signal).

Further, a molecular phylogenetic tree for the obtained novel sequence and the PIKK family molecules was prepared on the basis of the amino acid sequences, whereupon the cDNA consisting of the human cDNA clone KIAA0421 and its upstream region is closest to fruit-fly SMG-1 and C. elegans SMG-1, which are genes involved in the degradation of abnormal RNA, and thus was considered to encode human SMG-1. In this connection, human SMG-1 includes a sequence FRBH (FKBP12/rapamycin binding homology) having homology with the FKBP12/rapamycin binding site of FRAP/mTOR/RAFT1. Further, unlike other PIKK families, a long sequence of an approximately 1200 amino acids was inserted between the kinase domain and the FAT domain.

### Example 2

### Detection of mRNA of Human SMG-1 in Various Human Cell Lines by Northern Blotting

A total RNA was prepared from human cell lines HPB-ALL [Morikawa, S. et al., Int. J. Cancer, 21, 166 (1978)], HL-60 (CCL-240), U937 [Sundstrom, C. et al., Int. J. Cancer, 17, 565 (1976)], HepG2 (HB-8065), HeLa (CCL-2), PC3, A498, and 5873T using an RNA extraction kit (Quick Prep Total RNA extraction kit; Amersham Pharmacia Biotech) in accordance with the manual attached to the kit. The following blotting and hybrizing were performed in accordance with the document [Sugiyama, JBC, 275, 1095-1104, (2000)]. More particularly, the RNAs were electrophoresed, and then transferred to a polyamide membrane (Hybond; Amersham Pharmacia Biotech). The 5′-side fragment (corresponding to the base sequence consisting of the 6255th to 7048th bases in the base sequence of SEQ ID NO: 1) of the cDNA clone KIAA0421 of human SMG-1 was labeled using a Multiprime DNA Labelling System (Amersham Pharmacia Biotech) in accordance with the manual attached to the kit and using [α-³²P]dCTP (220 TBq/mmol; Amersham Pharmacia Biotech). The polyamide membrane to which the RNA has been transferred was hybridized with the labeled cDNA fragment as a probe, and was washed with 0.1×SSC [1.67 mmol/L sodium chloride and 1.67 mmol/L sodium citrate (pH7.0)]-0.1% sodium dodecyl sulfate (SDS) at 60° C. (30 minutes) three times, and then the signal was detected by autoradiography.

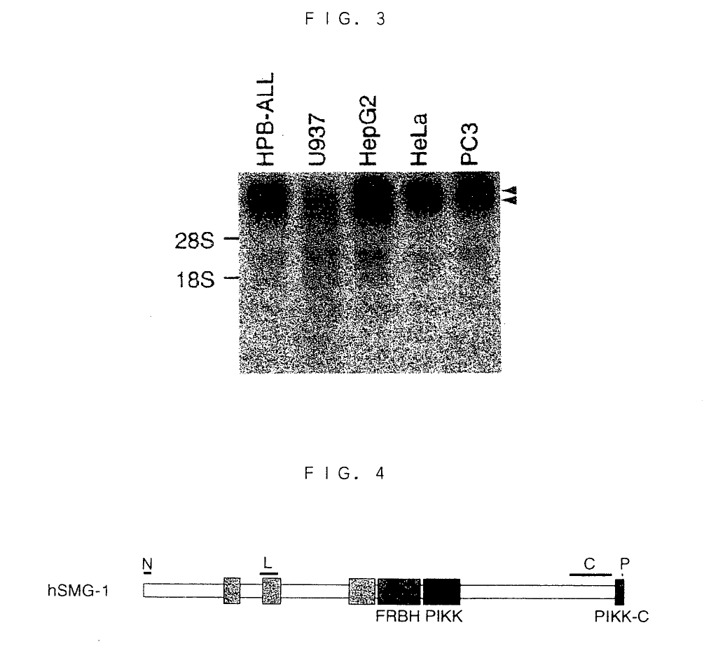

The results of autoradiography for HPB-ALL, U937, HepG2, HeLa, and PC3 are shown in FIG. 3. In FIG. 3, “28S” and “18S” show the electrophoresis positions of the 28S libosome RNA and 18S libosome RNA, respectively. As shown in FIG. 3, the two bands of mRNA of human SMG-1 shown by the arrows were detected. Further, in all remaining human cell lines (A549 and 293T), two bands were similarly detected (data not shown). Therefore, it was considered that two types,of lengths of mRNAs were transcribed from the human SMG-1 gene.

### Example 3

### Mapping of Human Chromosome by Fluorescent in Situ Hybridization (FISH) Method

FISH mapping was performed in accordance with the document [Izumi et al., JCB, 143, 95-106 (1998)]. More particularly, lymphocytes isolated from human blood were cultured, using a medium MEM (Minimal Essential Medium) to which 10% fetal bovine serum and phytohemagglutinin were added, at 37° C. for 68 to 72 hours. To the lymphocytes cultured while synchronizing the cell cycle, 0.18 mg/mL bromodeoxyuridine (BrdU; Sigma Aldrich) was added to be incorporated into the cells. The cells were washed three times with a serum-free medium, and then were recultured using an MEM containing 2.5 mg/mL thymidine (Sigma Aldrich) at 37° C. for 6 hours. The cells were collected and a slide was prepared by the standard method of a hyposmotic treatment, fixation, and air drying.

As the FISH probe, the cDNA clone KIAA0421 of human SMG-1 (full-length) was biotinylated using biotinylated DATP and a BioNick Labelling Kit (Life Technologies) at 15° C. for 1 hour [Heng HH et al., Proc. Natl. Acad. Sci. USA, 89, 9509-9513 (1992)]. In situ hybridization and its detection were performed in accordance with the method of the documents [Heng HH et al., Proc. Natl. Acad. Sci. USA, 89, 9509 (1992); Heng HH and Tsui LC, Chromosoma, 102, 325 (1993)]. Simply explained, the slide was heated at 55° C. for 1 hour (i.e., a ribonuclease treatment), then the slide was treated at 70° C. for 2 minutes using 2×SSC [33.3 mmol/L sodium chloride and 33.3 mmol/L sodium citrate (pH7.0)] containing 70% formaldehyde to denature the chromosomes, and dehydrated by ethanol. The probe was placed on the slide of the denatured chromosomes to perform hybridization overnight, and then the slide was washed and applied to the detection system. A signal appeared on the 16th chromosome, whereby it was found that the human SMG-1 gene is located on the 16th chromosome (16p12).

### Example 4

### Preparation of Antibody for Human SMG-1

Anti-human SMG-1 antiserum P1, antiserum C3, antiserum L1, antiserum L2, antiserum N1, and antiserum N2 were prepared by immunizing rabbits (New Zealand White) using the following immunogen together with adjuvants. As the adjuvants, Titer Max Gold (CytRx) was used for antiserum LT and antiserum NT, and Freund's adjuvant (Wako Pure Chemicals) was used for antisera other than antiserum LT and antiserum NT.

As the immunogen for antiserum PI, a peptide consisting of 15 amino acids corresponding to the C-terminus of human SMG-1 and bonded with keyhole limpet hemocyanin (KLH) was used. The peptide has an amino acid sequence wherein the cysteine residue was added to the N-terminus of the amino acid sequence of SEQ ID NO: 7 (CDNLAQLYEGWTAWV; i.e., the sequence consisting of the 3644th to 3657th amino acid residues in the amino acid sequence of SEQ ID NO: 2).

To prepare antiserum C3, a 1.4 kb MscI-MscI fragment (corresponding to the base sequence consisting of the 7641st to 9186th bases in the base sequence of SEQ ID NO: 1, and covering a half of the kinase insertion region at the C-terminal side) of the human SMG-1 cDNA of clone KIAA0421 was inserted into the SmaI site of the vector pGEX6P-3 (Amersham Pharmacia Biotech) for expressing a fusion protein with glutathione S-transferase (GST). E. coli BL21 was transformed with the plasmid to express the C-terminal fragment [corresponding to the amino acid sequence consisting of the 3076th to 3542nd amino acid residues in the human SMG-1 amino acid sequence (amino acid sequence of SEQ ID NO: 2)] of human SMG-1, as a fusion protein (molecular weight=approximately 70 kDa) with GST. The fusion protein produced in E. coli formed insoluble inclusion bodies. The purified inclusion bodies were dissolved in 1×SDS sample buffer [100 mmol/L TrisHCl (pH6.8), 2% SDS, 6% β-mercaptoethanol (β-ME), 10% glycerol, and 0.01% Bromophenol Blue]. SDS polyacryl amide gel electrophoresis (SDS-PAGE) was performed, and then the 70 kDa protein band was cut from the gel, finely pulverized, and used as the immunogen.

To prepare antiserum L1 and antiserum L2, similarly as the case of antiserum C3, an approximately 600 bp of cDNA fragment (corresponding to the base sequence consisting of the 2917th to 3505th bases in the base sequence of SEQ ID NO: 1) of the clone Liver33 was cut out and inserted into the vector pGEX6P-1 (Amersham Pharmacia Biotech) for expressing a fusion protein with GST. E. coli BL21 was transformed with the plasmid to express a human SMG-1 fragment (corresponding to the amino acid sequence consisting of the 864th to 1059th amino acid residues in the amino acid sequence of SEQ ID NO: 2) as a fusion protein (molecular weight=approximately 50 kDa) with GST. This fusion protein produced in E. coli was also insoluble, and thus the immunogen was prepared in a manner similar to the case of preparing the immunogen of antiserum C3.

To prepare antiserum N1 and antiserum N2, an approximately 0.7 kbp of SmaI-HincII fragment (corresponding to the base sequence consisting of the 306th to 645th bases in the base sequence of SEQ ID NO: 1) derived from the clone AI005513 was inserted into the vector pGEX-6P (Amersham Pharmacia Biotech) for expressing a fusion protein with GST. The produced recombinant protein was purified from E. coli by the standard glutathione beads method, and was used as the immunogen.

In FIG. 4, the antigen sites are schematically shown. In FIG. 4, the regions (CR1 to CR6 in FIG. 2) with a high homology with C. elegans SMG-1 are shown by gray or black squares. Further, in FIG. 4, “FRBH” means a sequence having homology with the FKBP12/rapamycin binding site (FKBP12/rapamycin binding homology), “PIKK” means a phosphatidyl inositol kinase (PIK) related kinase, and “PIKK-C” means a carboxyl terminal portion of the PIKK catalytic region. Further, the letters “N”, “L”, “C”, and “P” mean the antigen sites used for preparing antisera N1 and N2, antisera L1 and L2, antiserum C3, and antiserum P1, respectively.

### Example 5

### Detection of SMG-1 Protein in Various Animal Cells or Various Animal Tissues

(1) Detection of SMG-1 Protein in Various Animal Cell lysates by Western Blotting

HeLa cells were cultured in Dulbecco's modified Eagle's medium (DMEM) containing 7% fetal bovine serum, and were ultrasonicated in a lysis buffer F [20 mmol/L Tris-HCl (pH7.5), 0.25 mmol/L sucrose, 1.2 mmol/L EGTA, 20 mmol/L β-mercapto ethanol, 1 mmol/L sodium orthovanadate, 1 mmol/L sodium pyrophosphate, 1 mmol/L sodium fluoride, 1% triton X-100, 0.5% nonidet P-40, 150 mmol/L NaCl, 1 mmol/L PMSF (phenylmethylsulfonyl fluoride), 10 μg/mL leupepsin, and 2 μg/mL aprotinin] to prepare a cell lysate.

Similarly, various animal cell lysates were prepared for various cell lines derived from human, simian, mouse, and rat. More particularly, as the human cell lines, HeLa (ATCC: CCL-2), 293 (ATCC: CCL1573), HepG2 (ATCC: HB-8065), Jurkat [Schuneider, U. et al., Int. J. Cancer, 19, 621-626 (1977)], U937 [Sundstrom, C. et al., Int. J. Cancer, 17, 565 (1976)], HL-60 [Collins, S. J. et al., Nature, 270, 347 (1977)], and HPB-ALL [Morikawa, S. et al., Int. J. Cancer, 21, 166 (1978) were used. As the simian cell line, COS1 (ATCC: CRL1650) was used. As the mouse cell lines, NIH3T3 (ATCC: CRL1658), C3H10T1/2 (ATCC: CCL226), and C2C12 were used. As the rat cell lines, 3Y1 [Samdineyer, S. et al, Cancer Res., 41, 830 (1981)] and L6 [Yaffe, D. et al., Proc. Natl. Acad. Sci. USA, 61, 477-483 (1968)] were used.

For the resulting various animal cell lysates (corresponding to 20 μg of protein), SDS-PAGE was performed at the gel concentrations of 5.5% and 12.5%, and then Western blotting was carried out using antiserum P1, antiserum C3, antiserum L1, antiserum L2, antiserum N1, and antiserum N2, and a preimmunized serum for control.

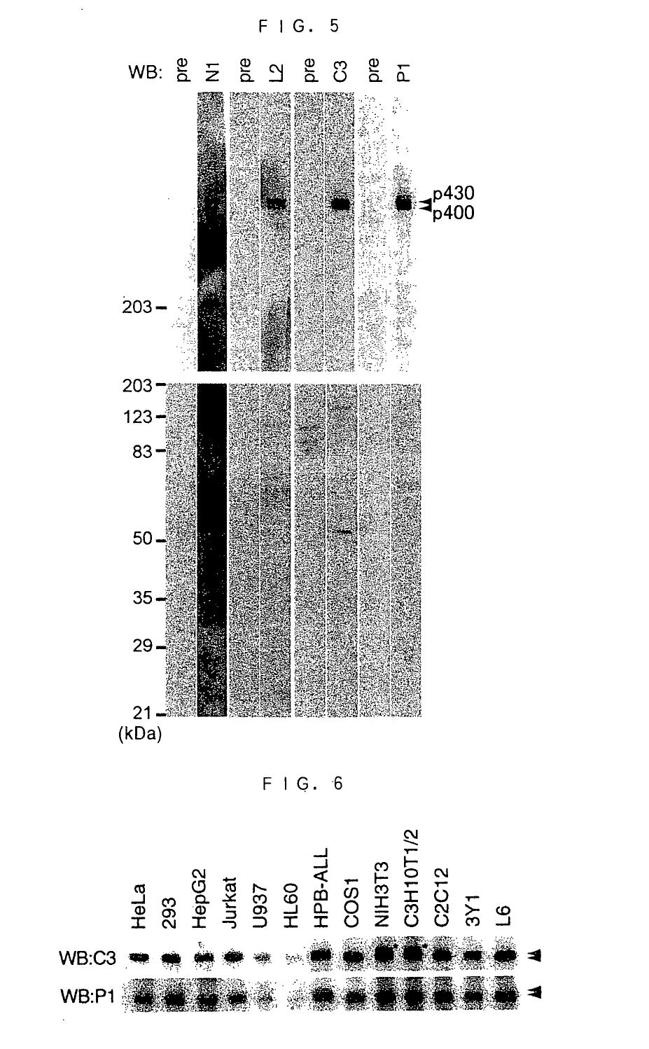

The results of use of antiserum P1, antiserum C3, antiserum L2, and antiserum N1 for the HeLa cell lysate are shown in FIG. 5. The results of use of antiserum P1 and antiserum C3 for various animal cell lysates are shown in FIG. 6.

In FIG. 5 and FIG. 6, “WB” means Western blotting. In FIG. 5, “pre” means the preimmunized serum. In FIG. 6, the arrow marks at the top in the “WB:C3” column or “WB:P1” column show p430, and the arrow marks at the bottom in the “WB:C3” column or “WB:P1” column show p400.

In all antisera other than antiserum N1 and antiserum N2, two protein bands of 400 kDa and 430 kDa were antiserum-specifically detected. Hereinafter, the SMG-1 protein having the molecular weight of 400 kDa will be sometimes referred to as p400, and the SMG-1 protein having the molecular weight of 430 kDa will be sometimes referred to as p430. Further, in the two mouse cell lines NIH3T3 and C3H10T1/2, a protein band of 460 kDa was detected in addition to the two bands of 400 kDa and 430 kDa.

On the other hand, in the antiserum N1 and antiserum N2, only the 430 kDa band was detected. Therefore, the 400 kDa band is considered to be an SMG-1 molecule in which an N-terminal portion of human SMG-1 is deleted.

To prove this hypothesis, the nucleotide sequence of the hSMG-1 cDNA was carefully examined, whereupon the presence of the methionine (Met) codon satisfying the translation start criteria of Kozak at the 129th position became clear. The estimated ORF starting from the 129th Met is a 396,040 Da protein consisting of 3529 amino acids. Therefore, it is probably believed that p400 is a product of the ORF starting from the 129th second methionine. (2) Detection of SMG-1 Protein by-Western Blotting in Cell Lysates Derived From Various Animal Tissues

With various tissues derived from rat and mouse, Western blotting was carried out using antiserum C3. Tissues were taken from animals by surgery, quickly frozen in liquid nitrogen, and powdered by crushing. Each powder was solubilized in a 1×SDS sample buffer, and then Western blotting was performed using 20 μg of protein from each tissue.

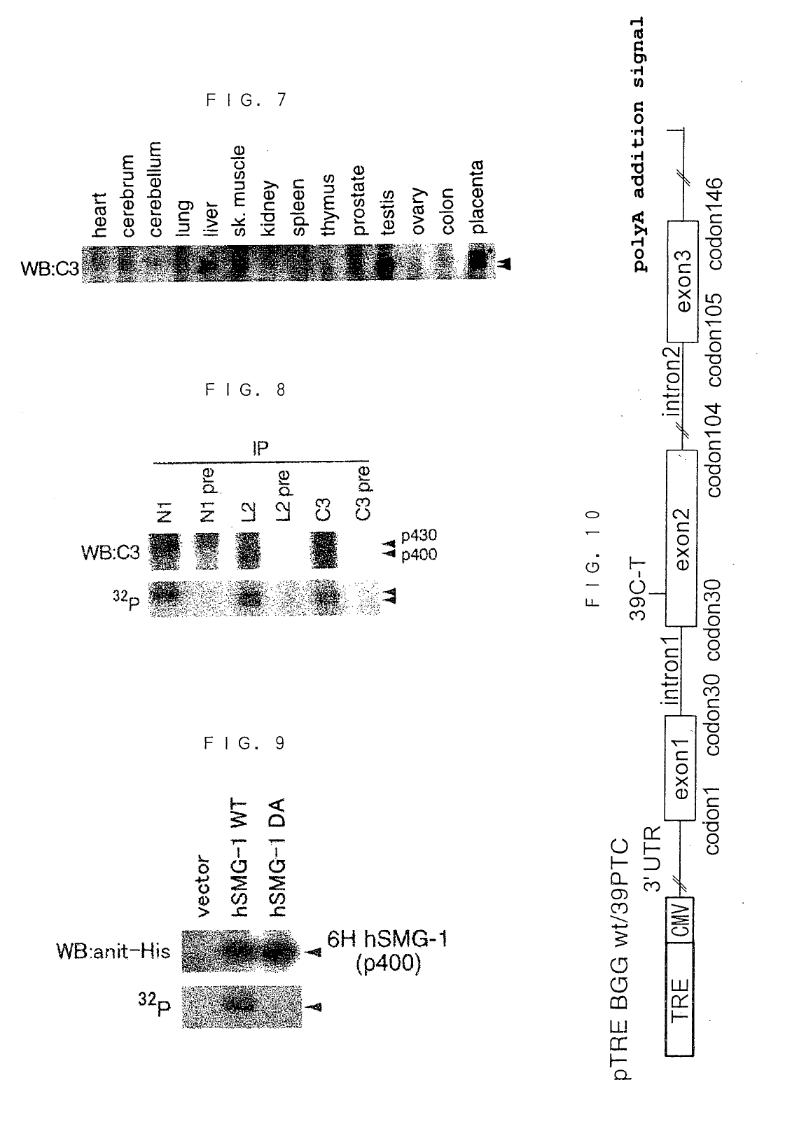

The results are shown in FIG. 7. In FIG. 7, “WB” means Western blotting, the upper arrow mark indicates p430, and the lower arrow mark indicates p400. As the rat tissues, the heart, cerebrum, cerebellum, lung, liver, skeletal muscle, kidney, spleen, thymus, prostate, ovary, testis, and colon were used, and as the mouse tissue, the placenta was used.

In all tissues, two bands of the 400 kDa protein (p400) and the 430 kDa protein (p430) were detected. In the mouse placenta, a 460 kDa protein band was also detected in addition to the two 400 kDa and 430 kDa bands, but the 460 kDa band was a nonspecific signal.

### Example 6

### Confirmation of Protein Kinase Activity of Human SMG-1 (Immunoprecipitate of Human HeLa Cell lysate by Anti-human SMG-1 Antiserum)

(1) Detection of SMG-1 Protein by Western Blotting in Immunoprecipitate of Human HeLa Cell Lysate by Various Human SMG-1 Antisera

The HeLa cell lysates obtained in a manner similar to that in the Example 5(1) were immunoprecipitated using antiserum N1, antiserum L2, and antiserum C3, and a preimmunized antiserum for control, respectively. The immunoprecipitation was performed by adding each antiserum to the cell lysate, allowing it to stand at 4° C. for 2 hours to form an immunocomplex, adding protein A sepharose CL-4B (Amersham Pharmacia Biotech), allowing it to stand for a further 2 hours to bond the immunocomplex, and recovering the protein A sepharose CL-4B by centrifugation. For each immunoprecipitate, SDS-PAGE was performed at a gel concentration of 5.5%, and Western blotting was performed using antiserum C3.

The results are shown in FIG. 8. In FIG. 8, “WB” means Western blotting, and “³²P” means the results of autoradiography in Example 6(2). Further, “pre” means the preimmunization serum, and “IP” means the immunoprecipitate. Further, the arrow at the top side in the “³²P” column shows p430, and the arrow at the bottom side in the “³²P” column shows p400.

As shown by the “WB:C3” column of FIG. 8, while two protein bands of 400 kDa and 430 kDa were detected by the antiserum C3 from the immunoprecipitate of antiserum L2 or antiserum C3, only the protein band of 430 kDa was detected by the antiserum C3 from the immunoprecipitate of the antiserum N1.

(2) Confirmation of Protein Kinase Activity of Immunoprecipitates of Human HeLa Cell Lysates by Various Human SMG-1 Antisera

The immunoprecipitates obtained in the Example 6(1) were washed with a lysis buffer F containing 0.25 mol/L LiCl, and then washed two times with a 1×kinase reaction buffer [10 mmol/L HEPES-KOH (pH7.5), 50 mmol/L β-glycerophosphoric acid, 50 mmol/L NaCl, 1 mmol/L dithiothreitol (DTT), and 10 mmol/L MnCl₂].

To each of the washed immunoprecipitates, 25 μL of 2×kinase reaction buffer (that is, two-fold concentrations of the above kinase reaction buffer) was added. The phosphorylation reaction was started by adding 10 mmol/L ATP and 370 kBq [γ-³²P] ATP (6000 Ci/mmol; Amersham Pharmacia Biotech) in equal amounts (25 μL) and continued, with occasional stirring, at 30° C. for 30 minutes. The final reaction amount was maintained at 50 μL, then 25 μL of a 4×SDS sample buffer was added to stop the reaction. SDS-PAGE was performed at gel concentrations of 5.5% and 12.5%, and then autoradiography was carried out to detect the phosphorylated proteins. The phosphorylation strength of each protein was measured by an Image Analyzer BAS2000 (Fuji Film).

The results are shown in FIG. 8. As shown in the “³²P” column of FIG. 8, in the immunoprecipitate by antiserum L2 or antiserum C3, phosphorylation proteins of the molecular weights 430 kDa and 400 kDa were detected. Proteins of the molecular weights 430 kDa and 400 kDa are believed to be human SMG-1, and thus it was found that human SMG-1 has an autophosphorylation activity.

### Example 7

### Expression of Fusion Protein of Human SMG-1 Protein Fragment and One-Amino-Acid-Substituented Mutant

In this example, expression vectors were prepared for expressing (1) a fusion protein (hereinafter referred to as “6H-hSMG-1”) of the human SMG-1 protein partial fragment having the amino acid sequence consisting of the 107th to 3657th amino acids in the amino acid sequence of SEQ ID NO: 2, and the His tag consisting of the amino acid sequence of SEQ ID NO: 8 [including six continuous histidine (His) residues] and (2) a kinase-deficient mutant [hereinafter referred to as “6H-hSMG-1(DA)”] in which the asparatic acid (D) corresponding to the 2331st asparatic acid in the amino acid sequence of SEQ ID NO: 2 in the 6H-hSMG-1 is replaced with alanine (A).

(1) Construction of Vector for Expression of Fusion Protein (6H-hSMG-1) of Human SMG-1 Protein Fragment and His Tag

An expression vector for expressing 6H-hSMG-1 was constructed by the following procedure.

The cDNA clone including a part (corresponding to the amino acid sequence consisting of the 107th to 3657th amino acids in the amino acid sequence of SEQ ID NO: 2) of the full-length of the hSMG-1 cDNA was digested by restriction enzymes HpaI and XhoI, and the 11 kbp DNA fragment was purified. The DNA fragment was inserted into the SmaI/XhoI site of an expression vector SR6H [a modified SRD vector having a base sequence encoding the His tag upstream of the multicloning site (MCS)] to obtain a vector SR6H-hSMG-1 for expressing the recombinant human SMG-1.

(2) Construction of Vector for Expressing One-Amino-Acid-Substituented Mutant [6H-hSMG-1(DA)] of 6H-hSMG-1

Next, a vector SR6H-hSMG-1 (DA) for expressing 6H-hSMG-1 (DA) was obtained by using the above expression vector SR6H-hSMG-1 and a commercially available kit (Chameleon Mutagenesis Kit, Stratagen).

(3) Confirmation of Expression of 6H-hSMG-1 and 6H-hSMG-1(DA) and Protein Kinase Activity in Vitro

After 293T cells were cultured using Dulbecco's modified Eagle's medium (DMEM; GibcoBRL), the cells were transfected with the expression vector SR6H-hSMG-1 prepared in Example 7(1) or the expression vector SR6H-hSMG-1(DA) prepared in Example 7(2). In this connection, as a control, transfection was also performed using the vector SR6H. After two days from the transfection, the cells were collected and lysed with the lysis buffer F.

Except for using an anti-polyhistidine antibody (His-Tag; Novagen), immunoprecipitation of each cell lysate was carried out in accordance with the procedure described in Example 6(1), and then the protein kinase activity in each of the resulting immunoprecipitates was measured in accordance with the procedure described in the Example 6(2). Further, Western blotting was also performed using the immunoprecipitates obtained by the immunoprecipitation.

The results are shown in FIG. 9. In FIG. 9, “WB:anti-His” shows the results of Western blotting by the anti-polyhistidine antibody, and “³²P” shows the results of autoradiography. Further, “vector” means the results in the case of use of the vector SR6H (control), “hSMG-1 WT” means the results in the case of use of the vector SR6H-hSMG-1, and “hSMG-1 DA” means the results in the case of use of the vector SR6H-hSMG-1 (DA). Further, the arrow mark in the “³²P” column shows 6H-hSMG-1.

As shown in FIG. 9, both 6H-hSMG-1 and 6H-hSMG-1(DA) were immunoprecipitated by the anti-polyhistidine antibody. Further, It was shown that the asparatic acid in the hSMG-1 corresponding to the 2331st asparatic acid in the amino acid sequence of SEQ ID NO: 2 (corresponding to the 2475th asparatic acid known to be essential for the kinase activity in ATR) is necessary for the kinase activity. As shown in FIG. 9, 6H-hSMG-1 obtained by the immunoprecipitation exhibits a mobility of approximately 400 kDa, and has a distinctive kinase activity. These results clearly show that 6H-hSMG-1 has a distinctive autophosphorylation activity.

### Example 8

### Confirmation of Involvement of SMG-1 in PTC Dependent Degradation of β-globin mRNA

(1) Construction of Reporter Gene Plasmid

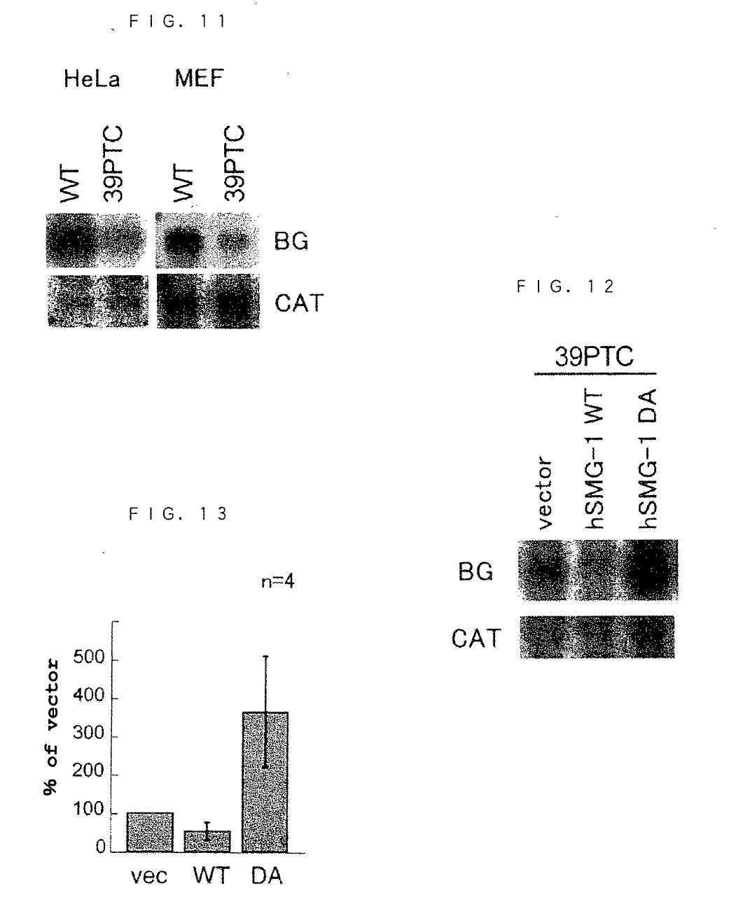

It was confirmed that, in C. elegans, seven types of smg genes are involved in NMD. The inventor made the unexpected discovery that a novel member of the PIKK family exhibits a similarity in overall sequence to C. elegans SMG-1, and thereby decided to investigate whether or not hSMG-1 is involved in the NMD of mammals. To this end, a reporter gene (FIG. 10) having a gene sequence with or without a PTC at the 39th codon of human β-globin (BGG) arranged downstream of the CMV promoter was constructed as follows. In this construction, the CMV promoter is under the control of the upstream tetracycline-responsive element (TRE) sequence. Further, when introduced into a cell line having a plasmid pTet OFF, the transcription from this reporter gene is stopped specifically and quickly in the presence of tetracycline or its derivative (doxycycline). In FIG. 10, an exon is shown by a square, and an intron is shown by a straight line.

To prepare a reporter gene plasmid PTRE BGG WT (PTC is absent at the 39th codon of BGG), a human β-globin gene fragment was amplified from a human gene library (Clonetech) by PCR, and was inserted into a PTRE vector (Clonetech). Further, a nonsense mutation of the human β-globin gene at the codon 39 was induced by the standard procedure to produce a reporter gene plasmid pTRE BGG PTC (PTC is present at the 39th codon of BGG).

(2) Evaluation of Amount of Accumulation of Reporter mRNA by Northern Blotting

A cell line HeLa Tet-OFF (Clonetech) or a cell line MEF Tet-OFF (Clonetech) was transfected with the reporter plasmid BGG-WT or the reporter plasmid BGG-39PTC prepared in the Example 8(1) together with a CAT plasmid as the internal standard, and was incubated in the absence of doxycycline, and then the accumulation of the BGG mRNA was evaluated by Northern blotting.

More particularly, as a transfection reagent, polyfectin (QIAGEN) was used in the case of the cell line HeLa Tet-OFF, and effectin (QIAGEN) was used in the case of the cell line MEF Tet-OFF. After 24 hours from the transfection, cells were re-inoculated in six 10 cm dishes and cultured in the absence of doxycycline for further 24 hours. The transcription from the reporter was stopped by adding 50 ng/mL of doxycycline, the cells were collected at the periods of 0 hour, 0.5 hour, 1 hour, or 3 hours, and then each of the total RNA was isolated. The amounts of BGG mRNA and CAT mRNA from equal amounts (2 μg) of cells were evaluated by Northern blotting using a BGG probe and a CAT probe.

The results are shown in FIG. 11. In FIG. 11, “WT” means the results of the case of using the reporter plasmid BGG-WT, and “39PTC” means the results of the case of use of the reporter plasmid BGG-39PTC. Further, “BG” means the results obtained by the BGG probe, and “CAT” means the results obtained by the CAT probe.

As shown in FIG. 11, in both cell lines, the accumulation of mRNA of BGG-WT (that is, BGG without PTC) was more abundant than the accumulation of BGG-39PTC (that is, BGG with PTC at the 39 position).

(3) Confirmation of Effect of 6H-hSMG-1 and 6H-hSMG-1(DA) on Accumulation of Reporter mRNA

The procedure in Example 8(2) was repeated except for transfecting either the expression vector SR6H-hSMG-1 prepared in the Example 7(1) or the expression vector SR6H-hSMG-1(DA) prepared in the Example 7(2) at the same time.

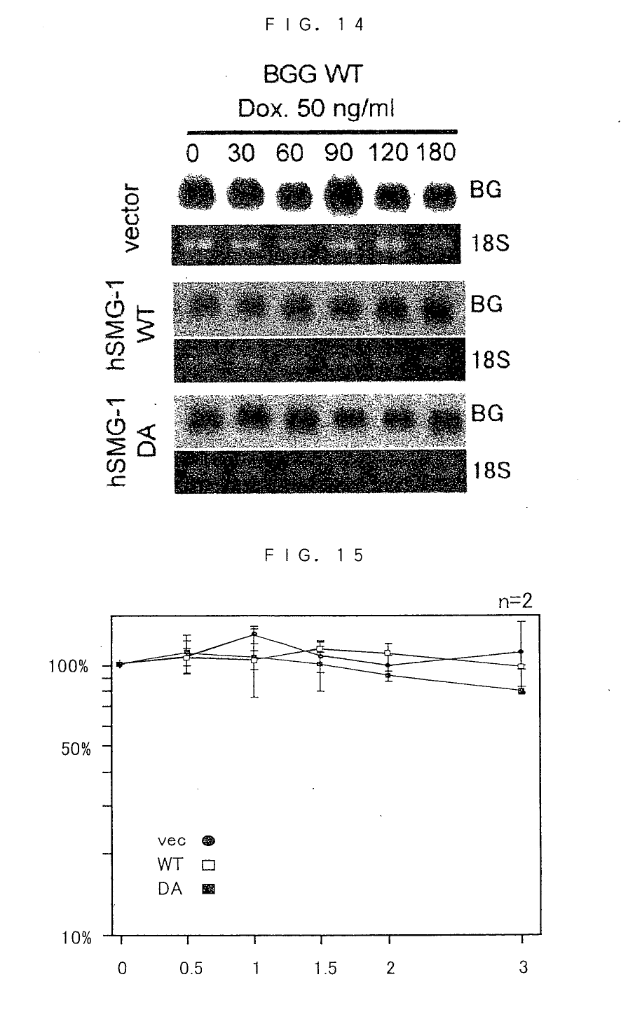

The results relating to BGG-39PTC in the HeLa Tet-OFF cells are shown in FIG. 12 and FIG. 13. In FIG. 12 and FIG. 13, “vector” or “vec” means the results in the case of use of the vector SR6H (control), “hSMG-1 WT” or “WT” means the results in the case of use of the vector SR6H-hSMG-1, and “hSMG-1 DA” or “DA” means the results in the case of use of the vector SR6H-hSMG-1 (DA). Further, “BG” means the results obtained by the BGG probe, and “CAT” means the results obtained by the CAT probe. Further “39PTC” means the results in the case of use of the reporter plasmid BGG-39PTC.

When 6H-hSMG-1 (DA) is overexpressed, the accumulation of the BGG-39PTC transcripts is amplified, while when 6H-hSMG-1 is overexpressed, the amount of stable state mRNA encoding BGG-39PTC is reduced, compared with introduction of the vector SR6H (control). These results provide powerful proof supporting the fact that hSMG-1 and its inherent protein kinase activity are involved in the PTC dependent decay of the BGG mRNA.

Next, to further confirm this fact, the effects of overexpression of 6H-hSMG-1 or 6H-hSMG-1(DA) in the half life of mRNA of BGG WT or BGG-39PTC were tested. The transcription from each of the BGG reporters was stopped by adding doxycycline to the incubator, the cells were collected at the predetermined periods (0 hour, 0.5 hour, 1 hour, 1.5 hours, 2 hours, and 3 hours), and then each of the BGG mRNA was measured.

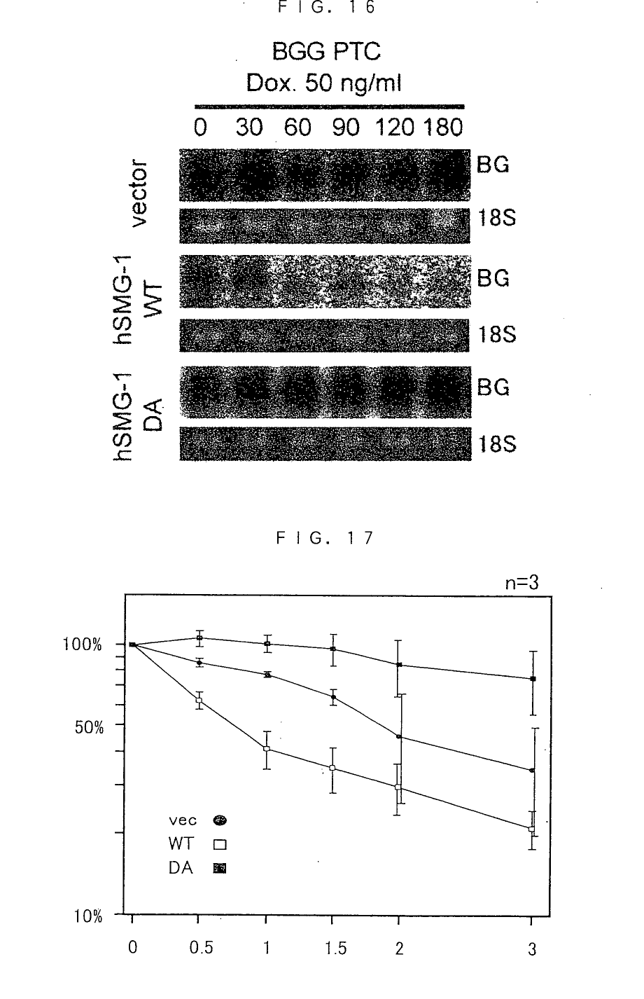

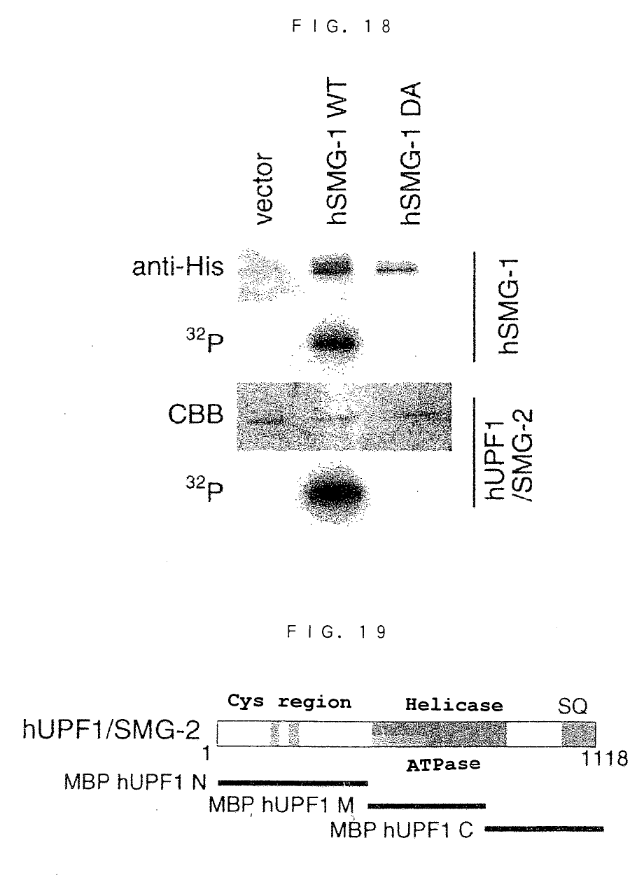

The results are shown in FIG. 14 to FIG. 17. In FIG. 14 to FIG. 17, “BGG WT” means the results in the case of use of the reporter plasmid BGG-WT, and “BGG PTC” means the results in the case of use of the reporter plasmid BGG-39PTC. Further, “vector” or “vec” means the results in the case of use of the vector SR6H (control), “hSMG-1 WT” or “WT” means the results in the case of use of the vector SR6H-hSMG-1, and “hSMG-1 DA” or “DA” means the results in the case of use of the vector SR6H-hSMG-1(DA). Further, “Dox.” means doxycycline, “BG” means BGG, and “18S” means 18S libosome RNA.

The half life of BGG WT appears to be extremely long, as already reported [Sun, X. et al., Proc. Natl. Acad. Sci. USA, 95, 10009-10014 (1998)], and further is not affected by the expression of either 6H-hSMG-1 or 6H-hSMG-1(DA). On the other hand, the half life of BGG-39PTC is greatly shortened by the overexpression of 6H-hSMG-1 and becomes longer due to the overexpression of 6H-hSMG-1(DA). When combining these results with the above results, it is clearly shown that 6H-hSMG-1 is involved in the decay of PTC-dependent BGG mRNA. Further, these results also show that the kinase activity of 6H-hSMG-1 plays an important role in the NMD of mammals.

### Example 9

### Phosphorylation of hUPF1/SMG-2 by 6H-hSMG-1 in Vitro

An experiment by Perlick [Perlick, H. A. et al., Proc. Natl. Acad. Sci. USA, 93, 10928-10932 (1996)] identified hUpf1 (a human homolog of yeast Upf1). Further, using a point mutation of the helicase domain of hUpf1, Sun et al. showed that hupf1 is involved in the NMD of mammals [Sun, X. et al., Proc. Natl. Acad. Sci. USA, 95, 10009-10014 (1998)]. More recently, Anderson confirmed that C. elegans SMG-2 protein is a homolog of Upfl in C. elegans [Page et al., Mol. Cell. Biol., 19, 5943-5951 (1999)]. SMG-2 is a phosphorylated protein. Further, of extreme importance, another six types of smg genes can be classified into two groups based on the effects of mutation in the phosphorylated state of SMG-2. In the mutants of smg-1, smg-2, and smg-3, SMG-2 in the phosphorylated state was not detected. In the mutants of smg-5, smg-6, and smg-7, phosphorylated SMG-2 was accumulated at a high level.

(1) Confirmation of Phosphorylation of Full-length hUpf1/SMG-2 Fusion Protein by 6H-hSMG-1

To test the possibility that hSMG-1 directly phosphorylates hUpf1/SMG-2, the HA tagged hUpf1/SMG-2 (hereinafter referred to as HA-hUpf1/SMG-2) was expressed in 293T cells, and HA-hUpf1/SMG-2 was purified.

More particularly, first, an expression vector for expressing HA-hUpf1/SMG-2 was prepared by the following procedure. That is, an SR vector [Hirai, S. et al., Oncogene, 12, 641-650 (1996)] was modified by inserting the HA tag at the multicloning site (MCS) and upstream thereof to obtain a vector SRHAI. Into the MCS of the obtained vector SRHAI, cDNA encoding the full-length of hUpf1/SMG-2 was inserted to obtain an expression vector SRHAI-hUpf1/SMG-2. More particularly, the vector SRHAI was cleaved by restriction enzyme BglII, and then blunted. Into the blunted vector, the cDNA clone KIAA0221, which had been cleaved by restriction enzymes XhoI and BlpI and then blunted, was inserted.

Then, 293T cells were transfected with the obtained expression vector SRHAI-hUpf1/SMG-2. Two days after the transfection, the cells were collected and lysed in the lysis buffer F. Anti-HA affinity beads (Rosche) were added to the lysate. After one hour, the beads were washed with the lysis buffer F three times and washed with a washing buffer [20 mmol/L Tris-HCl (pH7.5), 0.1 mol/L NaCl, 0.1 mmol/L EDTA, and 0.05% Tween20] three times. The resulting washed beads were treated in the washing buffer containing 1 mg/mL HA peptide (YPYDVPDYA) at 37° C. to elute the binding protein. Next, dialysis in 1×PBS containing 10% glycerol and 1 mmol/L DTT was carried out to obtain HA-hUpf1/SMG-2.

On the other hand, 6H-hSMG-1 and 6H-hSMG-1 (DA) were purified from cDNA-transfected 293T cells transfected by the expression vector SR6H-hSMG-1 prepared in Example 7(1) or the expression vector SR6H-hSMG-1 (DA) prepared in Example 7(2) in accordance with the procedure described in Example 7(3).

The phosphorylation reaction was performed in accordance with the procedure described in Example 6(2), except for adding HA-hUpf1/SMG-2 prepared in Example 9(1) to the 2×kinase reaction buffer as a substrate.

The results are shown in FIG. 18. In FIG. 18, “vector” means the results in the case of use of the vector SR6H (control), “hSMG-1 WT” means the results in the case of use of the vector SR6H-hSMG-1, and “hSMG-1 DA” means the results in the case of use of the vector SR6H-hSMG-1 (DA). “anti-His” means the results of Western blotting by the anti-polyhistidine antibody, “³²P” means the results of autoradiography, and “CBB” means the results obtained by the Coomassie Brilliant Blue (CBB) staining.

As shown in FIG. 18, purified 6H-hSMG-1 phosphorylated HA-hUpf1/SMG-2. This suggests that, at least in the system using the purified substance, hUpf1/SMG-2 becomes a direct substrate of hSMG-1. Kinases belonging to the PIKK family phosphorylate the serine or threonine residue in the SQ or TQ motif [Kim, S. T. et al., J. Biol. Chem., 274, 37538-37543 (1999)]. Of interest, hUpf1/SMG-2 contains a repetition of the SQ motif in the C-terminal region [Page et al., Mol. Cell. Biol., 19, 5943-5951 (1999)]. Taking into consideration the fact that hSMG-1 encodes the kinase belonging to the PIKK family, this suggests that the SQ motif is the target of hSMG-1.

(2) Confirmation of Phosphorylation by 6H-hSMG-1 in Fusion Protein of hUpf1/SMG-2 Partial Fragment (1)

To confirm the above hypothesis, a series of maltose binding protein (MBP) fusion proteins containing the fragmentated hUpf1/SMG-2 was constructed and purified.

More particularly, three types of cDNA fragments cut from SRHAI-hUpf1/SMG-2 [prepared in Example 9(1)] containing cDNA encoding hUpf1/SMG-2, that is, a cDNA fragment (1.4 kbp, BgIII-Eco47III fragment, corresponding to the amino acid sequence consisting of the 1st to 462nd amino acids of hUpf1/SMG-2) encoding a partial fragment at the N-terminal side, a cDNA fragment (1.0 kbp, Eco47IH-Eco47II fragment, corresponding to the amino acid sequence consisting of the 463rd to 800th amino acids of hUpf1/SMG-2) encoding a partial fragment in the intermediate region, and a cDNA fragment (1.4 kbp, Eco47llI-BstZ17I fragment, corresponding to the amino acid sequence consisting of the 801st to 1118th amino acids of hUpf1/SMG-2) encoding a partial fragment at the C-terminal side, were inserted into a pMaI-c2 vector (New England Biolabs) to obtain the expression vectors PMBP-hSMG-2 N, pMBP-hSMG-2 M, and pMBP-hSMG-2 C, respectively.

The obtained MBP fusion proteins were all extremely insoluble in E. coli, and thus the recombinant proteins were purified from inclusion bodies as follows. That is, the collected cells were suspended in an ultrasonication buffer [50 mmol/L TrisHCl (pH8.0), 50 mmol/L NaCl, 1 mmol/L EDTA, 1 mmol/L DTT, and 1% triton X-100] containing 2 μg/mL aprotinin, 10 μg/mL leupeptin, 2 mmol/L PMSF, and 50 mmol/L benzamidine, and were ultrasonicated. Each precipitate (mostly inclusion bodies) obtained by centrifugation at 0000×g was washed with a washing solution (0.5% triton X-100 and 1 mmol/L EDTA) five times. The washed precipitate was suspended in a denaturation buffer [8 mol/L urea, 50 mmol/L TrisHCl (pH8.0), 1 mmol/L DTT, and 1 mmol/L EDTA], and allowed to stand at room temperature for 1 hour. The supernatant obtained by centrifugation at 10000×g was dialyzed for 1 hour in a denaturation buffer containing 4 mol/L urea, then was dialyzed for 1 hour in a denaturation buffer containing 2 mol/L urea, and further was dialyzed overnight in the ultrasonication buffer. MBP fusion proteins (i.e., the fusion proteins of the partial fragment of Upf1/SMG-2 at the N-terminal side, the partial fragment in the intermediate region, or the partial fragment at the C-terminal side, with MBP) renaturated by this treatment was recovered and purified using an amylose resin (New England Biolabs) in accordance with the attached manual.

The phosphorylation reaction was performed in accordance with the procedure described in Example 6(2), except for adding as a substrate each MBP fusion protein to the 2×kinase reaction buffer and using, as hSMG-1, 6H-hSMG-1 prepared in accordance with the procedure described in Example 7(3).

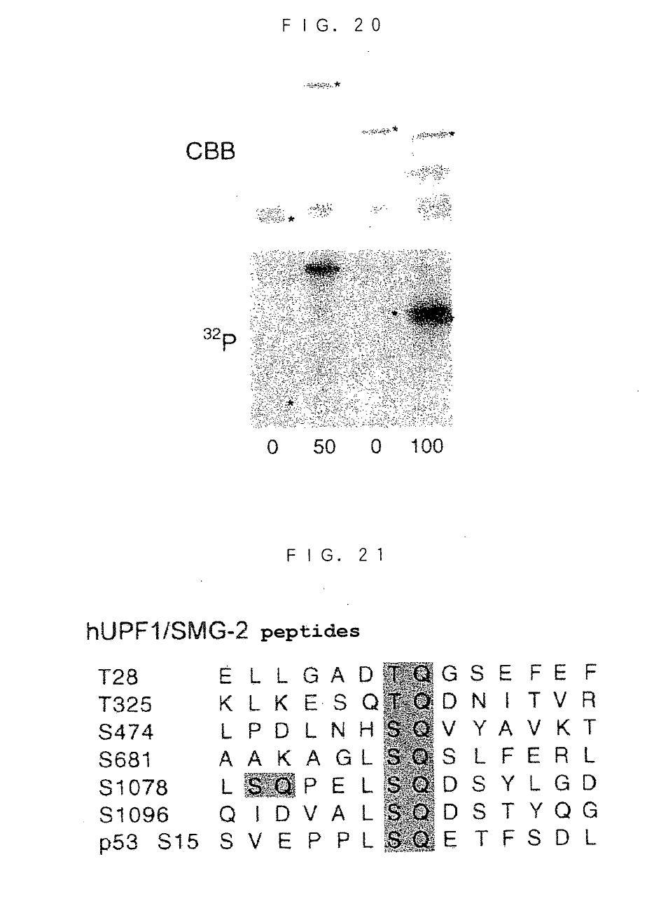

The results are shown in FIG. 19 and FIG. 20. In FIG. 20, “CBB” means the results by CBB staining, while “³²P” means the results of autoradiography. Further, the numerals shown under the autoradiograms are relative values when using the intensity of the autoradiogram in the fusion protein of pMBP-hSMG-2 C and MBP as 100.

As shown in FIG. 20, the fragments of hUpf1/SMG-2 at the C-terminal side and at the N-terminal side performed the role of good substrates for hSMG-1. The results of the fragment of hUpf1/SMG-2 at the C-terminal side being phosphorylated, taking into consideration the Page et al. report (that is, hUpf1/SMG-2 contains a repetition of the SQ motif at the C-terminal region), lead to the prediction that the SQ motif is phosphorylated. Further, as a result of the fragment of hUpf1/SMG-2 at the N-terminal side being phosphorylated, it is believed that there are plural SQ motifs at the N-terminal region and that there is a possibility that these sites are phosphorylated.

(3) Confirmation of Phosphorylation by 6H-hSMG-1 in Fusion Protein of hUpf1/SMG-2 Partial Fragment (2)

Next, to further clarify the above point, another series of GST fusion proteins was prepared. In this example, fusion proteins in which 14mer peptides consisting of the SQ or TQ deduced motifs in hUpf1/SMG-2 and the surrounding 12 amino acid residues were fused downstream of GST were prepared.

More particularly, each DNA encoding a 14mer peptide containing T28 (that is, the 28th threonine in hUpf1/SMG-2), T325 (that is, the 325th threonine), S474 (that is, the 474th serine), S681 (that is, the 681st serine), S1078 (that is, the 1078th serine), or S1096 (that is, the 1096th serine), or DNA encoding the 14mer peptide (control) containing S15 in the p53 protein (the 15th serine in the p53 protein) was inserted into a vector pGEX 6P (Amersham Pharmacia Biotech) to prepare each expression vector. Each GST fusion protein was purified from E. coli transformed with each expression vector by the standard glutathione beads method.

The amino acid sequences of the 14mer peptides are shown in FIG. 21. In FIG. 21, “T28” means the amino acid sequence of the 14mer peptide part in the fusion protein of GST and the 14mer peptide containing T28. Similarly, “T325”, “S474”, “S681”, “S1078”, and “S1096” mean the amino acid sequences of the 14mer peptide parts in the fusion proteins of GST and the 14mer peptides containing T325, S474, S681, S1078, and S1096, respectively. “p53 S15” means the amino acid sequence of the 14mer peptide part in the fusion protein of GST and the 14mer peptide (control) containing S15.

The phosphorylation reaction was performed in accordance with the procedure described in the Example 6(2), except for adding as the substrate each GST fusion protein to the 2×kinase reaction buffer and using, as hSMG-1, 6H-hSMG-1 prepared in accordance with the procedure described in Example 7(3).

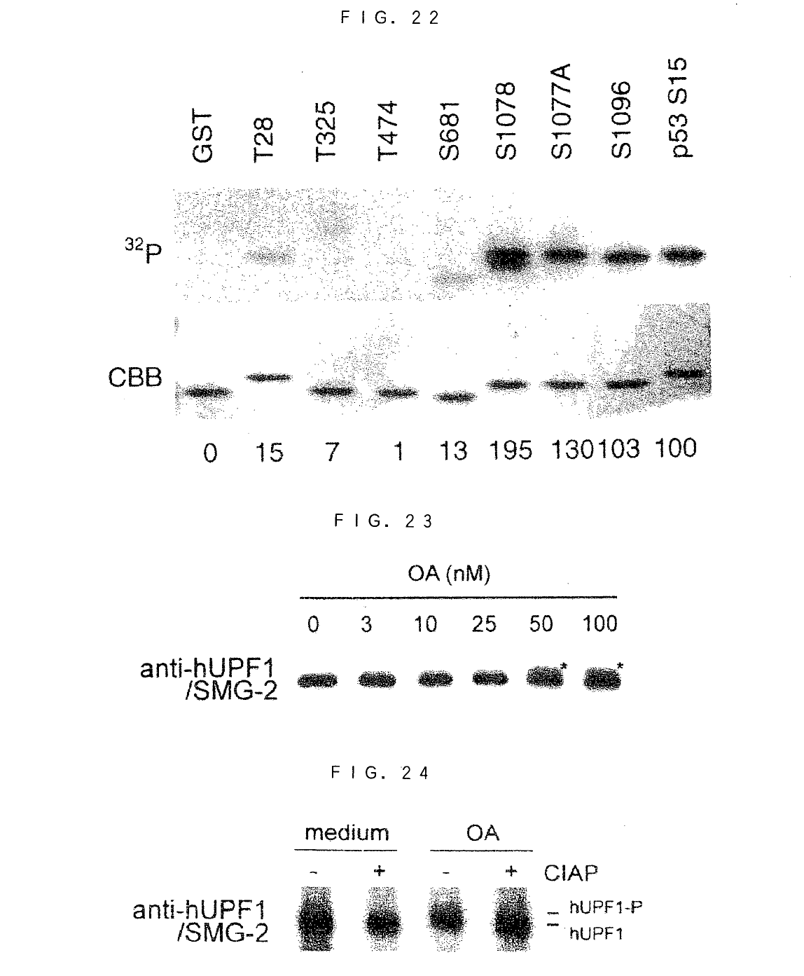

The results are shown in FIG. 22. In FIG. 22, “T28” means a fusion protein of the 14mer peptide including T28 and GST. Similarly, “T325”, “S474”, “S681”, “S1078”, and “S1096” mean fusion proteins of the 14mer peptides including T325, S474, S681, S1078, and S1096, and GST, and “p53 S15” means a fusion protein of the 14mer peptide (control) including S15 in the p53 protein and GST. “S1078A” means a point mutant in which the 1078th serine in “S1078” is replaced with alanine. Further, “CBB” means the results of CBB staining, while “³²P” means the results of autoradiography. Further, the numerals shown at the bottom of the autoradiograms are relative values in the case of using the strength of the autoradiogram in the fusion protein (p53 S15) of 14mer peptide including S15 in the p53 protein and GST as 100.

As shown in FIG. 22, the control construct encoding the SQ motif in the p53 protein was phosphorylated by hSMG-1. Further, the GST fusion protein including S1078 or the GST fusion protein including S1096 [hereinafter referred to as an hUpf1/SMG-2 fusion protein (S1096)] was efficiently phosphorylated by 6H-hSMG-1. These results establish that 6H-hSMG-1 phosphorylates the serine residues in S1078 and S1096 as the SQ motifs of hUpf1/SMG-2, at least in vitro.

### Example 10

### Confirmation of Phosphorylation of hUpf1/SMG-2 by SMG-1 in Cells

Considering the results obtained in the Example 9 (that is, the result that 6H-hSMG-1 phosphorylates hUpf1/SMG-2 in vitro) together with the results in the C. elegans smg genes, an interesting possibility is raised that hSMG-1 phosphorylates hUpf1/SMG-2 even in vivo and further, that the phosphorylation plays a fundamental role in NMD. As a first step for evaluating this possibility, the phosphorylation of hUpf1/SMG-2 was tested in vivo.

The HeLa cells were treated with various concentrations of okadaic acid (OA; Calbiochem) for 4.5 hours, and then were recovered and dissolved in the 1×SDS sample buffer. After 6% SDS-PAGE was performed, Western blotting using an anti-hUpf1/SMG-2 antibody was performed to determine the mobility shift of hUpf1/SMG-2.

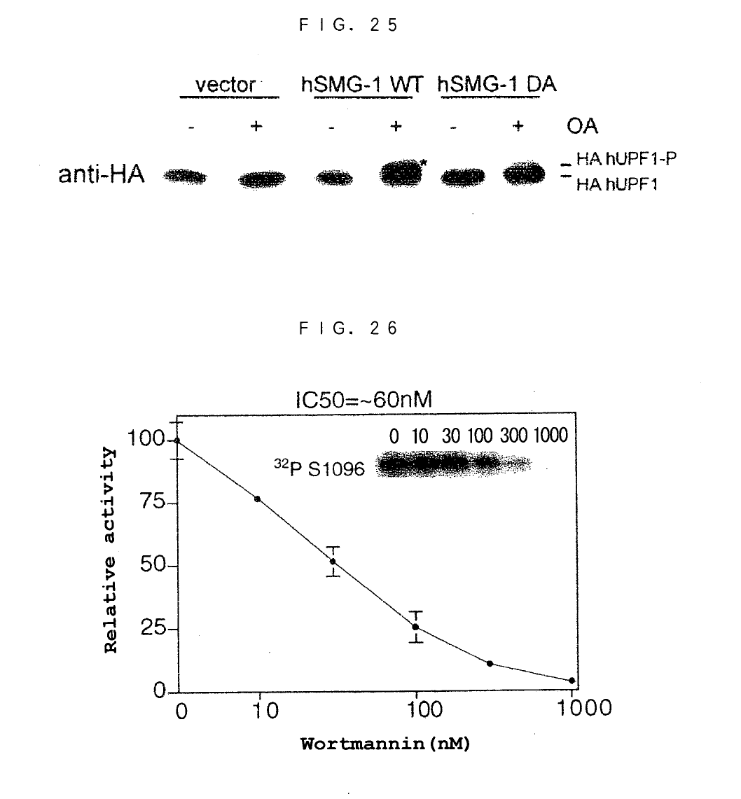

The results are shown in FIG. 23. When HeLa cells are treated with okadaic acid (OA), a phosphatase inhibitor, as a result, an upwardly shifted band of hUpf1/SMG-2 appears. In FIG. 23, the position of the shifted band is marked by an asterisk. Further, the “anti-hUPF1/SMG-2” in FIG. 23 means the results obtained by Western blotting using the anti-hUpf1/SMG-2 antibody.

To show that the upward shift of hUpf1/SMG-2 induced by OA arises due to phosphorylation, the immunopurified hUpf1/SMG-2 was treated with alkaline pbosphatase, then the mobility in SDS-PAGE was tested as follows.

That is, HeLa cells treated for 4.5 hours in the presence or absence (that is, only the medium) of 50 nmol/L okadaic acid were recovered, lysed in the lysis buffer F containing 1 μmol/L mycrocystin LR (Calbiochem) and 10 nmol/L okadaic acid, and then immunoprecipitated using an anti-hUpf1/SMG-2 serum. The reason why the mycrocystin and okadaic acid were added to the lysis buffer F was to prevent the once phosphorylated protein from being dephosphorylated during immunoprecipitation.

The immunoprecipitate was washed in the lysis buffer F and a dephosphorylation buffer [50 mmol/L Tris-HCl (pH9.0) and 1 mmol/L MgCl₂], and then suspended in 50 μL of the dephosphorylation buffer. Calf intestine alkaline phosphatase (CIAP; Takara Shuzo) was added in an amount of 0 unit (that is, not added) or 60 units to start the reaction. The mixture was incubated at 37° C. for 1 hour, then the SDS sample buffer was added to stop the reaction. After 6% SDS-PAGE was performed, the mobility shift of hUpf1/SMG-2 was determined by Western blotting using the anti-Upf1/SMG-2 antibody.

The results are shown in FIG. 24. In FIG. 24, “OA” means the results in the case of using the immunoprecipitate derived from cells treated with okadaic acid, while “medium” means the results in the case of using the immunoprecipitate derived from cells in the absence of okadaic acid. Further, “anti-hUPF1/SMG-2” means the results obtained by Western blotting using the anti-hUpf1/SMG-2 antibody. Further, “hUPF1-P” means phosphorylated hUpf1/SMG-2, while “hUPF1” means unphosphorylated hUpf1/SMG-2.

The upwardly shifted band disappeared in the case of treating the immunoprecipitate by phosphatase (CIAP). This shows that the upward shift of hUpf1/SMG-2 occurring due to the OA treatment is phosphorylation.

Next, to analyze the overexpressed hUpf1/SMG-2, 293T cells were transfected by the expression vector SRHAI-hUpf1/SMG-2 for expressing HA-hUpf1/SMG-2 prepared in Example 9(1) and the expression vector SR6H-hSMG-1 or vector SR6H-hSMG-1 (DA) prepared in Example 7(1). The cells were cultured for 4 hours in the presence or absence of 50 nmol/L okadaic acid. The cells were recovered and then dissolved in the 1×SDS sample buffer. The mobility shift of hUpf1/SMG-2 was determined by the Western blotting using an anti-HA antibody (12CA5; Boehringer).

The results are shown in FIG. 25. In FIG. 25, “vector” means the results when using the vector SR6H (control), “hSMG-1 WT” means the results when using the vector SR6H-hSMG-1, and “hSMG-1 DA” means the results when using the vector SR6H-hSMG-1 (DA). Further, “anti-HA” means the results of Western blotting using the anti-HA antibody. Further, “HA hUPF1-P” means phosphorylated HA-hUpf1/SMG-2, while “HA hUPF1” means unphosphorylated HA-hUpf1/SMG-2. In FIG. 25, the position of the shifted HA-hUpf1/SMG-2 is marked by an asterisk.

In a manner similar to the case of only the vector SR6H (control), when overexpressing 6H-hSMG-1 (DA), no OA-induced upward shift of the exogenous HA tagged hUpf1/SMG-2 was observed. However, when 6H-hSMG-1 was overexpressed, the OA-induced upward shift of the HA tagged hUpf1/SMG-2 was greatly amplified.

### Example 11

### Identification of Inhibitor using 6H-hSMG-1 Protein Kinase Activity as Indicator

From past research into the PIKK family, inhibitors acting in this family of kinases are identified. As the identified inhibitors, for example, wortmannin [Sarkaria, S. N. et al., Cancer Res., 58, 4375-4382 (1998)] and caffeine [Sarkaria, S. N. et al., Cancer Res., 59, 4375-4382 (1999)] may be mentioned. Next, to evaluate the role of hSMG-1 in NMD in mammals and to evaluate the potential strategy of specific inhibition of NMD by pharmacological operations on cell, hUpf1/SMG-2 fusion protein (S1096) prepared in Example 9(3) [that is, fusion protein in which the 14mer peptide including the 1096th serine (S1096) is fused downstream of GST] was used as the endogenous substrate, to evaluate the effects of these inhibitors in the hSMG-1 kinase activity.

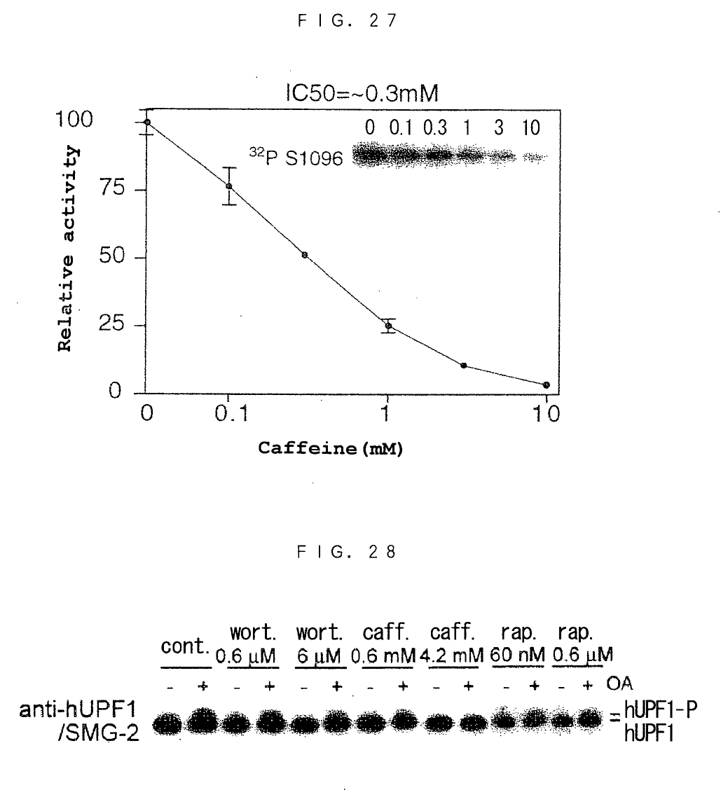

More particularly, 6H-hSMG-1 was prepared in accordance with the procedure described in Example 7(3). In the presence of various concentrations of wortmannin or caffeine shown in FIG. 26 and FIG. 27, the hUpf1/SMG-2 fusion protein (S1096) prepared in Example 9(3) was used as the substrate, to perform an in vitro kinase assay. That is, the phosphorylation was performed in accordance with the procedure described in Example 6(2), except for adding the hUpf1/SMG-2 fusion protein (S1096) and wortmannin or caffeine to the 2×kinase reaction buffer and using, as hSMG-1, 6H-hSMG-1 prepared in accordance with the procedure described in Example 7(3).

The results in the case of useing wortmannin are shown in FIG. 26, while the results in the case of useing caffeine are shown in FIG. 27. As shown in FIG. 26 and FIG. 27, both wortmannin and caffeine inhibited the kinase activity of 6H-hSMG-1 by IC50 values of approximately 60 nmol/L and 0.3 mmol/L, respectively. On the other hand, rapamycin did not inhibit hSMG-1 in the presence of purified recombinant FKBP12 (data not shown).

### Example 12

### Confirmation of SMG-1 Inhibitor Inhibiting Phosphorylation of hUpf1/SMG-2 in Cells

Further, the effects of the two types of hSMG-1inhibitor can also be tested in the phosphorylation of endogenous hUpf1/SMG-2 in HeLa cells.

HeLa cells were pretreated for 30 minutes in the presence or absence of various concentrations of wortmannin, caffeine, or rapamycin shown in FIG. 28. Next, the cells were treated for 4.5 hours in the presence of wortmannin, caffeine, or rapamycin and in the presence or absence of 50 nmol/L okadaic acid. Cell lysates were prepared and analyzed by Western blotting using the anti-Upf1/SMG-2 antibody.

The results are shown in FIG. 28. In FIG. 28, “anti-hUPF1/SMG-2” means the results obtained from Western blotting using the anti-hUpf1/SMG-2 antibody. Further, “cont.”, “wort.”, “caff.”, and “rap.” show the results of a control (that is, in the absence of wortmannin, caffeine, and rapamycin), the results in the presence of wortmannin, the results in the presence of caffeine, and the results in the presence of rapamycin, respectively. Further, “hUPF1-P” means phosphorylated hUpf1/SMG-2, while “hUPF1” means unphosphorylated hUpf1/SMG-2.

As shown in FIG. 28, wortmannin and caffeine both inhibited the upward shift of hUpf1/SMG-2 in HeLa cells, while rapamycin did not. This result matches with the results in the purified system (that is, the results of Example 11).

### Example 13

### Stabilization of Endogenous PTC mRNA by SMG-1 Inhibitor

(1) Stabilization of BGG Gene Product Containing Endogenous PTC by SMG-1 Inhibitor

If hSMG-1 plays an important role in the NMD of mammals, these hSMG-1 inhibitors should inhibit NMD. To test this, first, the reporter BGG systems utilizing the reporter plasmid BGG-WT or the reporter plasmid BGG-39 PTC prepared in Example 8(1) were applied.

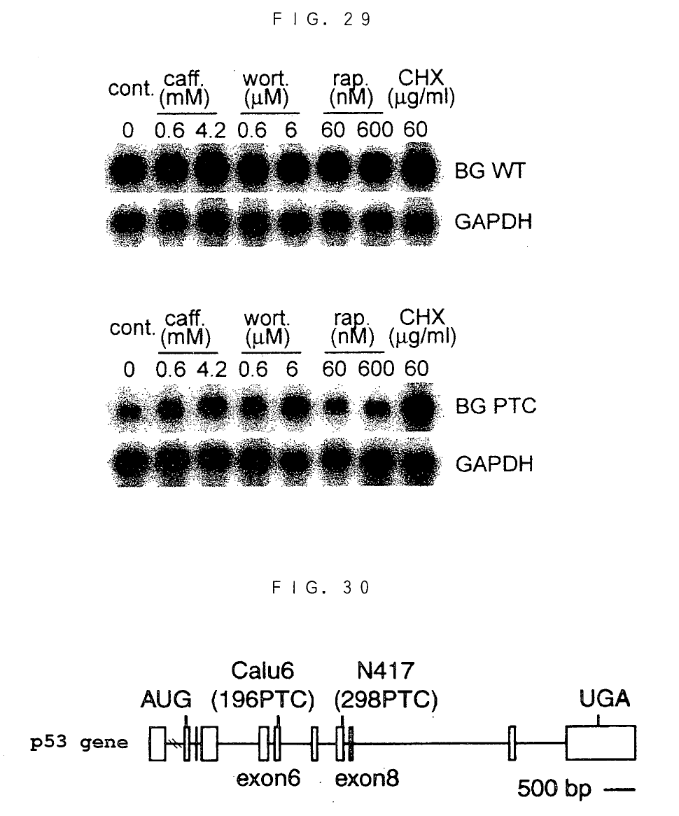

More particularly, MEF-Tet OFF cells were transfected with the reporter plasmid BGG-WT or the reporter plasmid BGG-39 PTC, and re-inoculated in eight dishes. The cells were then treated for 4.5 hours in the presence of 50 ng/ml doxycycline by various concentrations of caffeine (caff.), wortmannin (wort.), rapamycin (rap.), or cyclohexamide (CHX) shown in FIG. 29.

The Total RNA was analyzed by Northern blotting using the BGG probe. The results are shown in FIG. 29. In FIG. 29, “BG WT” means the results in the case of use of the reporter plasmid BGG-WT, “BG PTC” means the results in the case of use of the reporter plasmid BGG-39PTC, and “GAPDH” means the results in the case of use of the cDNA of glyceryl aldehyde-3-phosphate dehydrogenase as a probe. Further, “cont.”, “caff.”, “wort.”, “rap.”, and “CHX” show the results of the control (that is, in the absence of wortmannin, caffeine, rapamycin, and cyclohexamide), the results in the presence of caffeine, the results in the presence of wortmannin, the results in the presence of rapamycin, and the results in the presence of cyclohexamide, respectively.

As shown in FIG. 29, a protein synthesis inhibitor, CHX inhibited NMD. Further, BGG-39PTC mRNA (not BGG WT) was accumulated. This result matches the observations as described above. Of importance, the hSMG-1 inhibitors, that is, caffeine and wortmannin, resulted in the accumulation of BGG 39PTC. From this result, pharmacological proof supporting the assertion that hSMG-1 is involved in the NMD of mammals was obtained.

(2.) Stabilization of Endogenous PTC p53 Gene Product by SMG-1 Inhibitor

NMD rescues cells from the accumulation of potentially toxic proteins produced from PTC mRNA, but NMD often eliminates mRNAs encoding fragmentated proteins with residual activity capable of partially rescuing an impaired phenotype caused due to the mutation. Therefore, at least in the cases of several PTC mutations, it is possible to provide a novel method of treatment for rescuing the genetic disorders, by specifically inhibiting NMD.

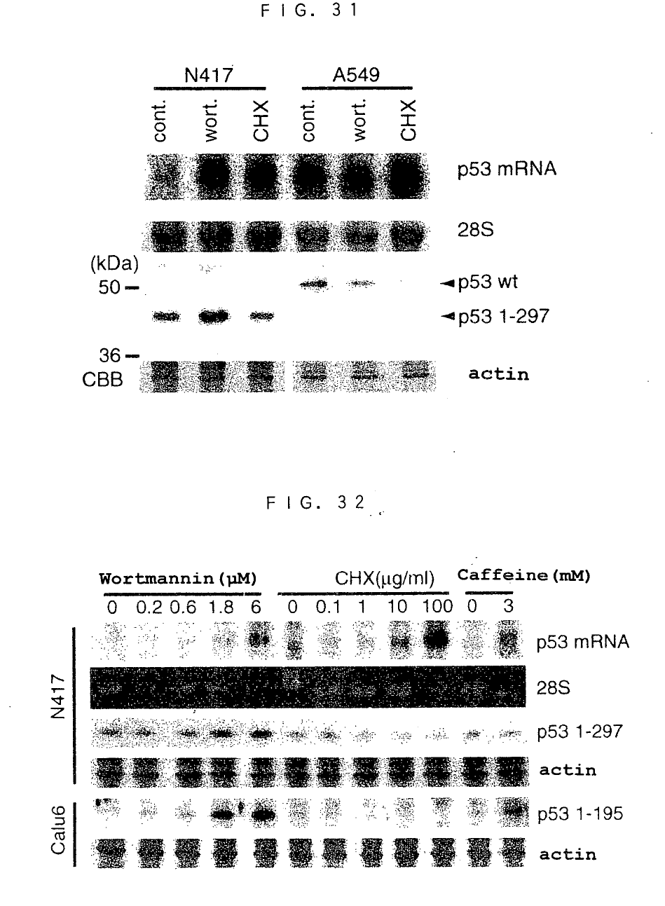

Next, as a first step for evaluating the possibilities of the method, the ability of the hSMG-1 inhibitors to specifically rescue the synthesis of fragmentated proteins was tested. As a model of a system for evaluating the possibility, the p53 gene was selected because cell lines having the mutation can be obtained. Two types of cell lines having PTCs, that is, Calu6 (lung adenocarcinoma cell line) including the PTC at the 196th codon and N417 (small cell lung adenocarcinoma cell line) including the PTC at the 1298th codon [Lehman TA, Cancer Research, 51, 4090-4096 (1991); Bodner SM, Oncogene, 7, 743-749 (1992)] were selected. The structure of the p53 gene and the PTC mutations of the cell lines Calu6 and N417 are schematically shown in FIG. 30. In FIG. 30, an exon is shown by a square.

The Calu6 and N417 cells, and the A549 cells [lung adenocarcinoma cell line; Lehman TA, cancer research, 51, 4090-4096 (1991)] as the control were treated in the presence or absence of 2 μmol/L wortmannin (wort.) or 50 pg/mL cyclohexamide (CHX) (cont.) for 4.5 hours, and then were recovered. The prepared cell lysates and total RNAs were analyzed by Northern blotting using a p53 probe and Western blotting using an anti-p53 antibody (DO-1; Calbiochem). A CBB image showing actin staining is also displayed.

The results in the N417 and A549 cells are shown in FIG. 31. In FIG. 31, “cont.”, “wort.”, and “CHX” show the results of the control, the results in the presence of wortmannin, and the results in the presence of cyclohexamide, respectively.

As a result of treatment of N417 cells by wortmannin, the p53 298PTC mRNA and the fragmentated p53 protein both increased, but in the control A549 cells, neither the mRNA nor the protein increased.

Further, the results in the case of treatment for 4.5 hours by various concentrations of wortmannin, cyclohexamide, or caffeine are shown in FIG. 32. In FIG. 32, “CHX” shows the results in the presence of cyclohexamide. The increase in the fragmentated p53 was also observed in the case of treatment of calu6 cells by an increased amount of wortmannin.

### Industrial Applicability

According to the polypeptide of the present invention, a convenient screening system for agents of treating and/or preventing a disease caused by one or more PTCs generated by a nonsense mutation can be provided. Further, the polynucleotide, expression vector, cell, and antibody of the present invention are useful in manufacturing the polypeptide of the present invention.

### Free Text in Sequence Listing

Features of “Artificial Sequence” are described in the numeric identifier <223> in the Sequence Listing. More particularly, the base sequence of SEQ ID NO: 8 in the Sequence Listing is a His tag containing six histidine residues.

Although the present invention has been described with reference to specific embodiments, various-changes and modifications obvious to those skilled in the art are possible without departing from the scope of the appended claims.

1. (1) A polypeptide comprising an amino acid sequence consisting of 129th to 3657th amino acids in the amino acid sequence of SEQ ID NO: 2, or (2) a polypeptide exhibiting an SMG-1 activity and comprising an amino acid sequence in which one or plural amino acids are deleted, substituted, and/or inserted at one or plural positions in an amino acid sequence consisting of 129th to 3657th amino acids in the amino acid sequence of SEQ ID NO: 2.

2. A polypeptide exhibiting an SMG-1 activity and comprising an amino acid sequence having a 90% or more homology, with an amino acid sequence consisting of 129th to 3657th amino acids in the amino acid sequence of SEQ ID NO: 2, with an amino acid sequence consisting of 1st to 3657th amino acids in the amino acid sequence of SEQ ID NO: 2, or with an amino acid sequence consisting of 107th to 3657th amino acids in the amino acid sequence of SEQ ID NO: 2.

3. A polypeptide consisting of the amino acid sequence of SEQ ID NO: 2.

4. A polynucleotide encoding the polypeptide according to any one of claims 1 to 3.

5. An expression vector comprising the polynucleotide according to claim 4.

6. A cell transfected with the expression vector according to claim 5.

7. An antibody or a fragment thereof, which binds to the polypeptide according to any one of claims 1 to 3.

8. A knock-out non-human animal wherein an expression of a gene encoding the polypeptide according to any one of claims 1 to 3 is partially or completely suppressed.

9. A method for screening a substance which modifies an SMG-1 activity of the polypeptide according to any one of claims 1 to 3, comprising the steps of:
bringing into contact (1) the polypeptide, (2) Upf1/SMG-2, a fragment thereof capable of being phosphorylated, or a fusion polypeptide comprising Upf1/SMG-2 or the fragment thereof, and (3) a substance to be tested; and carrying out phosphorylation under the conditions that the polypeptide is brought into contact with Upf1/SMG-2, the fragment thereof, or the fusion polypeptide, and analyzing whether or not Upf1/SMG-2, the fragment thereof, or the fusion polypeptide is phosphorylated.

10. A method for screening a substance which modifies an SMG-1 activity of the polypeptide according to any one of claims 1 to 3, comprising the steps of:
bringing (1) the polypeptide into contact with (2) a substance to be tested; and
carrying out phosphorylation under the conditions that the polypeptide is brought into contact with the substance to be tested, and analyzing whether or not the polypeptide is autophosphorylated.

11. An agent for suppressing nonsense-mediated mRNA decay, comprising, as an active ingredient, a substance which is obtained by the screening method according to claim 9 or 10 and modifies an SMG-1 activity of the polypeptide according to any one of claims 1 to 3.

12. An agent for suppressing nonsense-mediated mRNA decay, comprising as an active ingredient, an inhibitor of a phosphatidyl inositol kinase related kinase.

13. An agent for treating and/or preventing a disease caused by a premature translation termination codon generated by a nonsense mutation, comprising, as an active ingredient, a substance which is obtained by the screening method according to claim 9 or 10 and modifies an SMG-1 activity of the polypeptide according to any one of claims 1 to 3.

14. An agent for treating and/or preventing a disease caused by a premature translation termination codon generated by a nonsense mutation, comprising as an active ingredient, an inhibitor of a phosphatidyl inositol kinase related kinase.

15. An agent for suppressing nonsense, comprising as an active ingredient, (1) an SMG-1-acitivity-deficient mutant, or an inhibitor of a phosphatidyl inositol kinase related kinase, and (2) an aminoglycoside antibiotic.

16. An agent for suppressing nonsense, comprising, as an active ingredient, an SMG-1-acitivity-deficient mutant, or an inhibitor of a phosphatidyl inositol kinase related kinase.

17. An agent for promoting nonsense-mediated mRNA decay, comprising as an active ingredient, (1) the polypeptide according to any one of claims 1 to 3, (2) a substance which promotes an SMG-1 activity of the polypeptide, or (3) the polynucleotide according to claim 4.

18. A method for identifying a nonsense mutation point in a gene, comprising the steps of:
culturing a cell to be tested which is obtained from a subject to be tested and may contain a gene having a nonsense mutation by a premature translation termination codon, in the presence of an inhibitor of an SMG-1 activity; and
analyzing molecular weight of a polypeptide derived from the gene in the cultured cell.

19. A method for detecting a gene having a nonsense mutation, comprising the steps of:
culturing at least two groups of cells to be tested which are obtained from a subject to be tested and may contain a gene having a nonsense mutation by a premature translation termination codon, in the presence of an inhibitor of an SMG-1 activity and in the absence thereof, respectively; and detecting a presence or absence of the difference of an amount of mRNA derived from the gene in the cultured cells.
